(19) United States
(12) Patent Application Publication
(10) Pub. No.: US 2023/0293236 A1
(43) Pub. Date: Sep. 21, 2023

(54) DEVICE, METHOD AND COMPUTER PROGRAM PRODUCT FOR VALIDATING SURGICAL SIMULATION

(71) Applicant: Sony Group Corporation, Tokyo (JP)

(72) Inventors: Christopher WRIGHT, London (GB); Nicholas WALKER, London (GB); Naoyuki HIROTA, Tokyo (JP)

(73) Assignee: Sony Group Corporation, Tokyo (JP)

(21) Appl. No.: 18/014,759

(22) PCT Filed: Jul. 6, 2021
(86) PCT No.: PCT/JP2021/025430

§ 371 Date: Jan. 6, 2023

(30) Foreign Application Priority Data
Jul. 14, 2020 (EP) 20185663.0

(51) Int. Cl. A61B 34/10

(52) U.S. Cl. CPC A61B 34/10; A61B 34/25

(57) ABSTRACT

A device for validating a surgical simulation is provided in accordance with embodiments of the disclosure, the device including circuitry configured to: identify a portion of interest of a surgical event based on surgical information; provide an interactive surgical simulation to a network of surgeons, the interactive surgical simulation including the portion of interest of the surgical event; receive performance data from the network of surgeons, the performance data being indicative of actions taken by one or more surgeons in response to the interactive surgical simulation; and validate at least one of the portion of interest and/or the interactive surgical simulation based on the received performance data.

### TECHNICAL FIELD

The present disclosure relates to a device, method and computer program product for validating a surgical simulation.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

In recent years, significant technological developments in medical systems and equipment have been achieved. Computer assisted surgical systems, such as robotic surgical systems, now often work alongside a human surgeon during surgery. These computer assisted surgical systems include master-slave type robotic systems in which a human surgeon operates a master apparatus in order to control the operations of slave device during surgery.

In certain situations, it is advantageous to construct a surgical plan prior to performing a surgical procedure. The surgical plan may include information of certain steps which should be taken during the surgical procedure. The plan may also be adapted or reconfigured during consecutive stages of a surgical procedure. In particular, the surgical plan may also include information regarding certain tools or equipment which are required at specific stages during the surgical procedure. Accordingly, surgical plans improve the efficiency and effectiveness of the surgical procedure.

However, surgical environments and surgical procedures are inherently complex, often involving multiple independently moving components. It can, therefore, be difficult to predict the full range of possible scenarios which may occur during a surgical operation. This can cause an element of uncertainty to arise in the surgical plan.

Moreover, it can be difficult to adapt to changing conditions within a surgical procedure, particularly when an unexpected event arises leading to a number of distinct potential surgical outcomes.

It is an aim of the present disclosure to address these issues.

### SUMMARY

According to a first aspect of the disclosure, a device for validating a surgical simulation is provided, the device including circuitry configured to: identify a portion of interest of a surgical event based on surgical information; provide an interactive surgical simulation to a network of surgeons, the interactive surgical simulation including the portion of interest of the surgical event; receive performance data from the network of surgeons, the performance data being indicative of actions taken by one or more surgeons in response to the interactive surgical simulation; and validate at least one of the portion of interest and/or the interactive surgical simulation based on the received performance data.

According to a second aspect of the disclosure, a method of validating a surgical simulation is provided, the method comprising: identifying a portion of interest of a surgical event based on surgical information; providing an interactive surgical simulation to a network of surgeons, the interactive surgical simulation including the portion of interest of the surgical event; receiving performance data from the network of surgeons, the performance data being indicative of actions taken by one or more surgeons in response to the interactive surgical simulation; and validating at least one of the portion of interest and/or the interactive surgical simulation based on the received performance data.

According to a third aspect of the disclosure, a computer program product comprising instructions which, when the program is executed by a computer, cause the computer to perform a method of validating a surgical simulation is provided, the method comprising: identifying a portion of interest of a surgical event based on surgical information; providing an interactive surgical simulation to a network of surgeons, the interactive surgical simulation including the portion of interest of the surgical event; receiving performance data from the network of surgeons, the performance data being indicative of actions taken by one or more surgeons in response to the interactive surgical simulation; and validating at least one of the portion of interest and/or the interactive surgical simulation based on the received performance data.

According to embodiments of the disclosure, it is possible to efficiently identify and confirm the most likely and impactful events which may occur during a surgical procedure, thus enabling optimization of a surgical plan and facilitating interaction between human and robotic surgeons.

It will be appreciated that the present disclosure is not particularly limited to these advantageous technical effects. Other technical effects and advantages will become apparent to the skilled person when reading the disclosure.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 illustrates an apparatus or device which can be used in accordance with embodiments of the disclosure.

FIG. 2 illustrates a device according to embodiments of the disclosure.

FIG. 3 illustrates an example situation according to embodiments of the disclosure.

FIG. 4 illustrates an example event label tree in accordance with embodiments of the disclosure.

FIG. 5A illustrates an example training system in accordance with embodiments of the disclosure.

FIG. 5B illustrates an example training method in accordance with embodiments of the disclosure.

FIG. 6 illustrates an example surgical simulation according to embodiments of the disclosure.

FIG. 7 illustrates a method according to embodiments of the disclosure.

FIG. 8A illustrates an example implementation of a system in accordance with embodiments of the disclosure.

FIG. 8B illustrates an example method in accordance with embodiments of the disclosure.

FIG. 9 illustrates an example of a computer assisted surgery system according to embodiments of the disclosure.

FIG. 10 illustrates example of a computer assisted surgery system according to embodiments of the disclosure.

FIG. 11 illustrates an example of a computer assisted surgery system according to embodiments of the disclosure.

### DESCRIPTION OF EMBODIMENTS

Referring now to the drawings, wherein like reference numerals designate identical or corresponding parts throughout the several views.

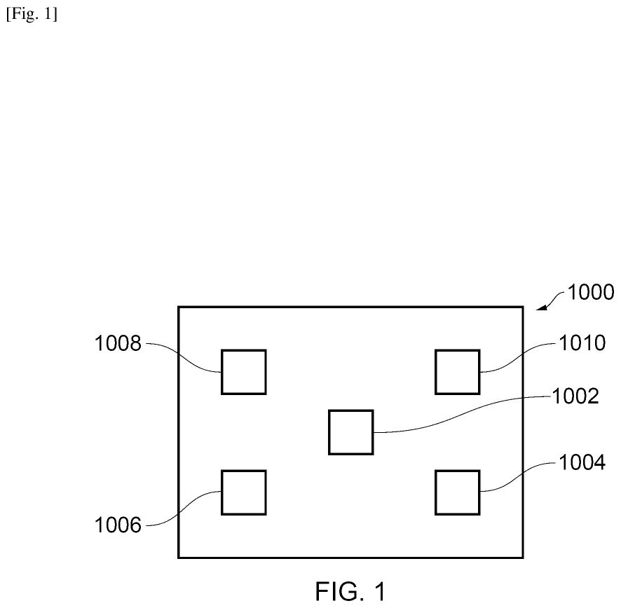

FIG. 1 illustrates an apparatus, system or device which can be used in accordance with embodiments of the disclosure. Typically, an apparatus 1000 according to embodiments of the disclosure is a computer device such as a personal computer or a terminal connected to a server. Indeed, in embodiments, the apparatus may also be a server. The apparatus 1000 is controlled using a microprocessor or other processing circuitry 1002. In some examples, the apparatus 1000 may be a portable computing device such as a mobile phone, laptop computer or tablet computing device. The processing circuitry 1002 may be a microprocessor carrying out computer instructions or may be an Application Specific Integrated Circuit. The computer instructions are stored on storage medium 1004 which maybe a magnetically readable medium, optically readable medium or solid state type circuitry. The storage medium 1004 may be integrated into the apparatus 1000 or may be separate to the apparatus 1000 and connected thereto using either a wired or wireless connection. The computer instructions may be embodied as computer software that contains computer readable code which, when loaded onto the processor circuitry 1002, configures the processor circuitry 1002 to perform a method according to embodiments of the disclosure.

Additionally, an optional user input device 1006 is shown connected to the processing circuitry 1002. The user input device 1006 may be a touch screen or may be a mouse or stylist type input device. The user input device 1006 may also be a keyboard or any combination of these devices.

A network connection 1008 may optionally be coupled to the processor circuitry 1002. The network connection 1008 may be a connection to a Local Area Network or a Wide Area Network such as the Internet or a Virtual Private Network or the like. The network connection 1008 may be connected to a server allowing the processor circuitry 1002 to communicate with another apparatus in order to obtain or provide relevant data. The network connection 1002 may be behind a firewall or some other form of network security.

Additionally, shown coupled to the processing circuitry 1002, is a display device 1010. The display device 1010, although shown integrated into the apparatus 1000, may additionally be separate to the apparatus 1000 and may be a monitor or some kind of device allowing the user to visualize the operation of the system. In addition, the display device 1010 may be a printer, projector or some other device allowing relevant information generated by the apparatus 1000 to be viewed by the user or by a third party.

As previously discussed, it is often necessary to produce a surgical plan prior to a surgical procedure. However, it can be difficult to produce surgical plans which are representative of realistic predictions for an upcoming procedure.

Consider an example situation whereby a surgeon (or a surgical team) is preparing for an upcoming surgical event or procedure. The surgeon will know certain details about the upcoming surgical procedure (e.g. the type of surgical procedure which must be performed). In particular, the surgeon may also know certain information about the patient (e.g. the age of the patient). In this situation, the surgical plan may include a development of the surgical steps which will be performed during the surgery. The surgical plan may also include a consideration of the tools and equipment which will be required at each stage of the surgical procedure. This may also include analysis of the steps which may be performed with the assistance of a robotic surgical device, for example.

However, in spite of detailed planning, it can be difficult for a surgeon to develop a comprehensive surgical plan prior to the surgical procedure. In part, this is due to the inherent complexity of surgical procedures; it can be difficult for a surgeon to predict the various complications which may arise during surgery. Moreover, the surgical plan developed by the surgeon is often biased by the surgeon's own training and experience, meaning that the surgeon may not consider all possible options for the upcoming surgical event. Finally, it can be difficult for the surgeon to understand the best way to utilize robotic surgical devices during a surgical procedure. This may lead to low efficiency of use of these robotic surgical devices.

The inventors have realized that pre-surgical planning may be improved by creating interactive surgical simulations of an upcoming surgical event, where data may be gathered from performance of a network of individual surgeons within the simulation to validate and improve the surgical simulation of the upcoming surgery. The validated simulation can be used to better advise a surgeon or surgical team in the production of a surgical plan prior to surgery, for example.

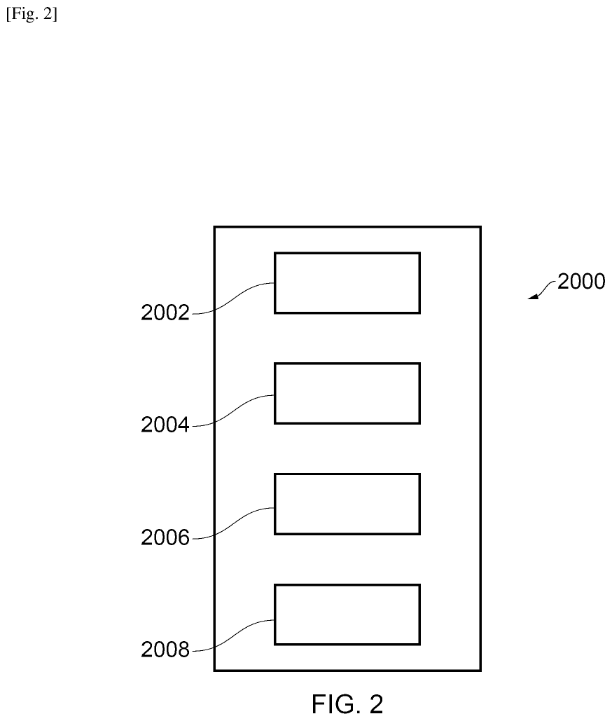

As such, in accordance with embodiments of the disclosure, a system, apparatus or device for validating a surgical simulation is provided. An illustration of a system (or apparatus or device) for validating a surgical simulation is provided in FIG. 2 of the present disclosure. The system 2000 includes an identifying unit 2002, a providing unit 2004, a receiving unit 2006 and a validation unit 2008. One or more of the identifying unit 2002, the providing unit 2004, the receiving unit 2006 and the validating unit 2008 may be implemented by a device such as device 1000 illustrated with reference to FIG. 1 of the present disclosure.

According to embodiments of the disclosure, the identifying unit 2002 is configured to identify a portion of interest of a surgical event based on surgical information. The providing unit 2004 is configured to provide an interactive surgical simulation to a network of surgeons, the interactive surgical simulation including the portion of interest of the surgical event. The receiving unit 2006 is configured to receive performance data from the network of surgeons, the performance data being indicative of actions taken by one or more surgeons in response to the interactive surgical simulation. Finally, the validating unit 2008 unit is configured to validate at least one of the portion of interest and/or the interactive surgical simulation based on the received performance data.

Accordingly, the system identifies certain points of interest in a surgical event and provides interactive surgical simulations to a network of surgeons. Performance data from the surgeons obtained within the interactive surgical simulation is then used in order to validate and improve the surgical simulation of the surgical event.

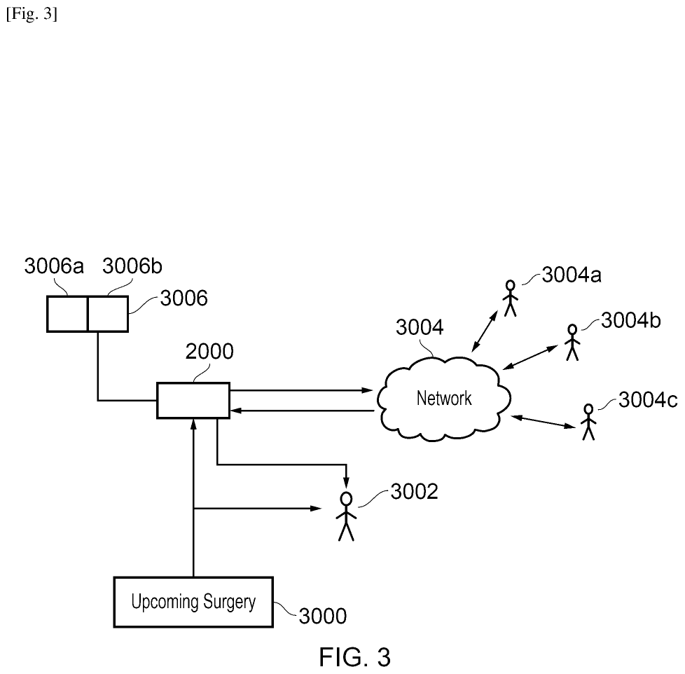

Consider now the example situation illustrated in FIG. 3 of the present disclosure.

In this example situation, a surgeon 3002 receives information of an upcoming surgery 3000. The details of the upcoming surgery 3000 may include information such as the type of surgery which is to be performed and the name of the patient for the upcoming surgery. Alternatively, the details of the upcoming surgery 3000 may include a unique identifier which can be used to identify the upcoming surgery. In this example, these details are also provided to system 2000 (being a system such as that illustrated with reference to FIG. 2 of the present disclosure).

System 2000 is configured to validate a surgical simulation in order to assist surgeon 3002 in the production of a surgical plan for the upcoming surgery 3000.

When the information regarding the upcoming surgery 3000 is received by system 2000, the system is configured to identify a portion of the upcoming surgery which may be of particular interest (e.g. a portion of the surgery which is of particular high risk or complexity). In this example, the portion of interest may be identified based upon information obtained from a database 3006. Such information may include details of previous surgical procedures which are similar to the upcoming surgery 3000; from the details of previous surgical procedures, portions of the previous surgical procedures which were particularly high risk or complex can be identified. This can then be used to predict risky or complex portions of the upcoming surgery (i.e. the portions of interest). Once the portions of interest have been identified, the system can provide an interactive surgical simulation to the network 3004, the interactive surgical simulation including at least a portion corresponding to the portion of interest which has been identified. That is, at least part of the interactive simulation is a simulation of the portion of interest in the upcoming surgery. Details regarding the provision of the interactive surgical simulation will be described in more detail below.

In this example, network 3004 may be a network such as the internet. The network 3004 may also be a local network (either wired or wireless). A number of surgeons 3004a, 3004b and 3004c are able to use electronic devices to connect to this network 3004. From the network 3004, the surgeons 3004a, 3004b and 3004c are able to receive the interactive surgical simulation of the upcoming surgery 3000. These surgeons are then able to attempt the interactive surgical simulation on their individual devices. Performance data regarding the manner in which the surgeons attempted the interactive surgical simulation (such as the decisions they made during the interactive surgical simulation) is then uploaded via the network 3004 to the system 2000.

It will be appreciated that the number of surgeons connected to the network 3004 (that is, the number of surgeons in the network of surgeons) is not particularly limited to the number illustrated in the example of FIG. 3. A much greater number of surgeons may be connected to the network 3004.

Once the system 2000 has received the performance data from the network 3004, the system 2000 can use that performance data to validate the interactive surgical simulation and/or the portion of interest. That is, for example, the performance data may indicate that an unexpected portion of the upcoming surgery caused the surgeons of the network of surgeons the most difficulty or confusion. In this case, said portion of the surgery can be updated as a portion of interest of the upcoming surgery 3000. Furthermore, the performance data may demonstrate that a certain action taken in response to an event within the surgical simulation is most favored by the networked surgeons and/or leads to the best outcome for the patient in the surgical simulation. System 2000 may then validate this action as the best action to take in the upcoming surgery 3000.

Once validated, the interactive surgical simulation may be provided to the surgeon 3002. In examples, this enables the surgeon 3002 to attempt the surgical simulation in order to practice for the upcoming surgery 3000. Furthermore, the validated interactive surgical simulation may enable the surgeon 3002 to understand the best action to take in the upcoming surgery, thus enabling the surgeon 3002 to produce an effective surgical plan for the upcoming surgery 3000.

Optionally, certain statistical information regarding the performance data received from the network and certain statistical information regarding the validated interactive surgical simulation may be provided to the surgeon by system 2000. This may enable the surgeon 3002 to understand the best configuration of surgical devices to use for the upcoming surgery 3000 (e.g. system 2000 may advise that 95% of networked surgeons used a certain surgical tool or robotic device at a given stage of the surgical procedure in the simulation).

Additionally, the system 2000 may also provide this information to a robotic control system (the robotic control system being used to control robotic surgical devices in the upcoming surgery 3000). This information enables the robotic control system to adjust its surgical plan prior to the surgical procedure in accordance with the information received from the network of surgeons. In some examples, the robotic control device may adapt its plan based on the validated surgical simulation so that fewer interventions by the operating surgeon (e.g. surgeon 3002) are likely to be required in the upcoming surgery 3000. This increases the efficiency of use of robotic devices during surgery.

Moreover, in some examples, a number of robotic control systems may also be connected to the network 3004, such that the robotic control systems may also provide performance data in response to the interactive surgical simulation (that is, as an alternative, or in addition to, the human surgeons 3004a, 3004b and 3004c). This enables the interactive surgical simulation to be validated based upon performance data of a number of different types of robotic control systems.

As such, validation of the surgical simulation in by the system 2000 enables identification and selection of the most likely and impactful events which may occur during a surgical procedure, thus optimizing the utility of the input from the network of surgeons. On this basis, it is possible to efficiently identify the most likely and impactful events which may occur during a surgical procedure, thus enabling optimization of a surgical plan and facilitating interaction between human and robotic surgeons.

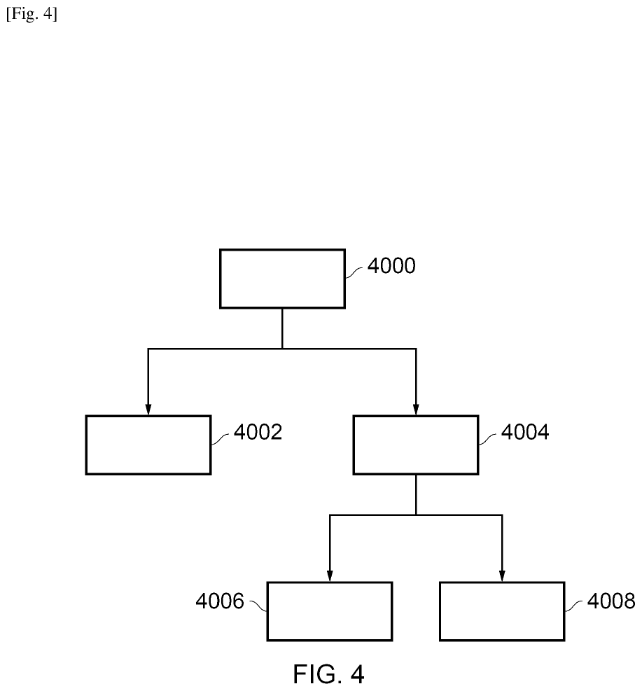

Further aspects of the system for validating a surgical simulation will be described in more detail with reference to FIGS. 4 to 6 of the present disclosure.

<Identification Unit>

As described above with reference to FIG. 2 of the present disclosure, the identification unit 2002 of system 2000 is configured to identify a portion of interest of a surgical event based on surgical information.

Information regarding the upcoming surgical event may be provided by the surgical information. In particular, the surgical information may include details of the upcoming surgery including the type of surgery (e.g. eye surgery), the operation identity (e.g. cataracts surgery), the system to be used during the surgery (e.g. surgical robot model information or the like). In addition to the information regarding the details of the upcoming surgery, the surgical information may also include information regarding the patient. This information may include patient electronic medical record data, patient pre-surgical scan data (e.g. X-ray or CT scan data), or the like.

Moreover, the surgical information may include information regarding past surgeries. Past surgical information may include information regarding actions taken by a surgeon in previous operation which is considered similar to the upcoming surgery (e.g. a surgery with a similar operation identity). For a group of past surgeries, this may be in the form of probabilities of different actions given a stimulus occurrence. That is, in the event of a bleed in a certain tissue area, a first surgeon may be 80% likely to cauterise the bleed themselves, and 20% likely to ask a second surgeon to perform the cauterisation on their behalf. These probabilities may be pre-calculated from assessments of past surgeries performed by the operating surgeon or surgeons.

In some examples, a trained model (such as a machine learning model), may use the information regarding past surgeries and the surgical information in order to identify the portions of interest in the upcoming surgery. These machine learning models are described in more detail below.

The past surgeon's success with different surgical actions may also form part of the surgical information. This data may be in the form of a percentage error rate with actions of a certain type based on automatic or manual flagging of serious errors or complications in past surgical performance data.

The surgical information is not limited to the above, and will vary in accordance with the type of surgery which is being performed.

In some examples, the surgical information may be provided directly to the identification unit 2002 of system 2000 (through user input, for example). That is, the surgeon 3002 may themselves provide information of the upcoming surgery 3000 to the identification unit 2002 of system 2000. Alternatively, the system 2000 may be configured to control one or more sensors and/or other devices in or to obtain at least a portion of the surgical information. These sensors may include patient monitoring sensors (such as blood pressure sensors), imaging sensors (configured to obtain an image of the surgical environment in which the surgery is to be performed) or the like.

In other examples, the surgical information (such as the information regarding past surgeries) may be stored internally within the system 2000. The operating surgeon for the upcoming surgery (e.g. surgeon 3002) would not need to input the surgical information to the identification unit 2002 in this case.

Moreover, in other examples, the surgical information may be stored externally to the system 2000. Consider the example illustrated in FIG. 3 of the present disclosure. In this example, a database 3006 is provided, the database storing the surgical information. Therefore, in this example situation, the surgical information required to identify a portion of interest in a surgical event may be obtained by the identification unit 2002 from the surgical database 3006.

The database 3006 may include a first portion 3006a storing details of the upcoming surgery and a second portion 3006b storing details of a previous surgical event. Additionally, a third portion (not shown) may be included in the database 3006 configured to store information regarding a current surgical event (such as a real time update of a surgical procedure).

The surgical information may be stored in a number of different formats depending on the type of surgical information and the manner by which that surgical information was originally obtained. That is, information of the upcoming surgery may be stored in the form of pre-surgical scans, patient medical records or the like. The surgical information may also be stored as image data, video data, surgical notes or the like.

Consider the past surgical data (being data of previous surgeries). Video data of past surgeries may be annotated by video processing software to label the events which occur in the video data. Past surgical data may therefore consist of video data sections associated with surgery type, operating surgeon identity, and/or semantic labels which are assigned to points in time or temporal sections within the video data. Generally, markers or identifiers within the surgical data may be considered to be ‘Event Labels’. These Event Labels may consist of, or identify, adverse events such as a bleed, surgeon error, or other event which negatively impacts the surgical outcome. Alternatively, the Event Labels may also include operating surgeon actions or events such as a decision to make an incision or a decision to apply suction.

Past surgical data may therefore be structured and grouped within the database (or other storage) according to these Event Labels and their sequential relationship within past surgeries.

FIG. 4 of the present disclosure illustrates an example tree of Event Labels which may be used in accordance with embodiments of the present disclosure. These Event Labels may correspond to certain annotations or flags provided on video data of a previous surgeries. In particular, across a collection of previous surgeries, each Event Label may represent a juncture in the surgery, where the operating surgeon made a decision regarding how to proceed.

In this example of FIG. 4, a first Event Label 4000 may be an event which is common in all the previous surgeries. That is, prior to the first Event Label 4000, there may be no divergence between the past surgical procedures. First Event Label 4000 therefore represents the first juncture in the flow of the previous surgeries. After the first Event Label 4000, the past surgical data may be structured according to decision taken by the operating surgeon at the first Event Label 4000. That is, in 60% of the past surgical data, the operating surgeon may make a first decision at Event Label 4000, which leads to a second Event Label 2. Alternatively, in the other 40% of the past surgeries, the operating surgeon may make a second decision at Event Label 4000, leading to a third Event Label 4004. Likewise, the third Event Label 4004 represents a further branch in the past surgical data, corresponding to whether a decision is made at the third Event Label 4004 which leads to a fourth Event Label 4006 or a fifth Event Label 4008.

In fact, there may be many decisions which can be taken by a surgeon at an Event Label, each leading to a separate branch within the past surgical data. That is, the present disclosure is not limited to a choice of a first option and a second option at each Event Label as illustrated in this example. There may be many more options available at each Event Label.

Structuring and grouping the past surgical data with Event Labels according to the decisions made by the operating surgeon as described above enables efficient storage and retrieval of the past surgical data in the storage database. Furthermore, use of Event Labels in this manner enables commonality amongst the past surgical data to be readily identified.

Returning now to FIG. 2 of the present disclosure, the identification unit 2002 may further be configured to use the surgical information (both upcoming surgical information and past surgical information) in order to identify a portion of interest in the upcoming surgery. The upcoming surgery may be a surgical procedure (or a portion therefore) which a human and/or robotic surgeon will perform on a patient. The identification unit may therefore identify a particular portion (or section) of the upcoming surgical event that is considered to be the most complex or risky for the surgeon to perform. Consider an example whereby a surgeon is going to perform colonoscopy on a patient. Based on the surgical information, the identification unit 2002 may recognise that certain portions of the colonoscopy procedure are inherently more complex and/or pose a higher risk to the patient (having a higher risk of causing a bleed, for example). These more critical portions are identified as portions of interest in the upcoming surgery.

As such, the identification unit 2002 system uses the surgical information to predict the critical points within an upcoming surgery where there is the greatest risk or uncertainty. This enables the interactive surgical simulation to be directed to the parts of the upcoming surgery where a validated simulation will be of most additional advantage to the operating surgeon.

It is noted that the portions of an upcoming surgery which are identified as the portions of interest (that is, the critical portions of the upcoming surgery) may vary between patients and are not limited solely to a predication based on the outcome of previous surgical events. That is, the upcoming surgical information (being information of the upcoming surgery) can be used to identify the portions of interest in the upcoming surgery. As an example, analysis of a pre-surgical scan (such as a CT scan or the like) may enable the system 2000 to analyse that a certain portion of the upcoming surgery will be more complex for an individual patient. Alternatively, or in addition, this prediction of the portions of interest may also vary based upon individual medical measurements or records of the patient (e.g. certain aspects of the upcoming surgery may be more complex for a patient with high blood pressures, for example).

Therefore, based on the surgical information, the identification unit 2002 identifies one or more portions of interest in the upcoming surgery.

In some examples, the portion of interest may be defined in the same format as Event Labels (indicating certain events of interest with surgical information).

In examples, the identification unit 2002 may extract past surgical data from a past surgical database which matches with upcoming surgical data on a set of pre-defined key parameters (such as type of surgery, age of patient and the like). These matching parameters may also include other data such as operating surgeon data (e.g. an experience level of the surgeon performing the surgery).

An optional step of extracting past surgical data based on the operating surgeon data may be useful in surgical scenarios where the parameters of the operating surgeon are important to the determination of the possible complications which may arise during a surgical procedure. For example, surgeon skill level or experience may have a high impact on the number and type of complications which may arise for more difficult surgeries and could therefore be used to predict likely points of interest within the surgery.

Once the past surgical data has been extracted, it may then be statistically analysed to determine the likelihood of different events (or Event Labels) occurring for the upcoming surgery. This likelihood may be defined as the proportion of matched past surgeries which contain each potential Event Label. Probabilities for individual events corresponding to each Event Label may, in some examples, be defined on a 0-1 scale. However, the present disclosure is not particularly limited in this regard.

Once the probability of each Event Label occurring in the upcoming surgery has been determined (based on the surgical information) the probability may then, in some examples, be averaged with a pre-defined ‘significance value’ for each Event Label. Again, in some examples, this significance value may also be a value of between 0 and 1, with most serious events (requiring complex intervention and/or corresponding to an undesirable outcome for the patient) being afforded a significance value of 1. For example, an Event Label of a bleed occurring in the upper colon may have a likelihood of 0.1 (occurring in 1 in 10 of the past surgeries) and a significance of 0.5 (requiring immediate intervention by the operating surgeon).

The probability of the event occurring and the seriousness of the event if that event does occur (the significance value) may be combined to produce a ‘criticality value’. In examples, this may be achieved by an average of the two values. In the example of a bleed in the upper colon, this would provide a criticality value of 0.3.

Other mathematical functions, such as multiplication, may be used in order to identify the combined ‘criticality value’; the present disclosure is not particularly limited to an averaging of the likelihood and significance value.

On this basis, a number of the most critical (being the most likely and significant) events may then be selected by the identification unit 2002 as the portions of interest in the upcoming surgery. For example, the identification unit 2002 may select the Event Labels with the top five highest criticality values as the top five portions of interest in the upcoming surgery.

In some examples, only the most critical Event Label may be selected as the portion of interest. In other examples, the number may be much higher than the top five most critical.

It will be appreciated that the identification unit 2002 is not particularly limited to determining the portion of interest based on the above description of the criticality value. Rather, any suitable method of identifying the portion of interest may be used by the identification unit 2002 in this regard, provided that the identifying unit 2002 identifies the portion of interest based upon the surgical information.

<Providing Unit>

As described above with reference to FIG. 2 of the present disclosure, the providing unit 2004 is configured to provide an interactive surgical simulation to a network of surgeons, the interactive surgical simulation including the portion of interest of the surgical event.

In examples, the interactive simulation may be an interactive video where the networked surgeons (such as 3004a, 3004b and 3004c described with reference to FIG. 3 of the present disclosure) may select actions from a set of pre-programmed options within the interactive simulation. As noted above, the interactive simulation includes at least the portion of interest (or portions of interest) which has been identified by the identification unit 2002. That is, if, based on the surgical information, the identification unit identifies a certain event (such as a certain type of incision) is a portion of interest of the upcoming surgery, the provision unit will provide an interactive simulation which includes at least the stage of performing that incision during the surgical procedure. This enables system 2000 to explore how the networked surgeons respond to the interactive simulation of the portion of interest of the upcoming surgery (i.e. the critical event within the upcoming surgery).

In some examples, the interactive simulation may include one or more ‘options’ which can be selected by a surgeon within the interactive simulation (e.g. corresponding to each potential decision which can be made at a certain Event Label or juncture in the interactive surgical simulation). Depending on the action taken by the surgeon within the surgical simulation a different progression of the interactive surgical simulation will then be experienced by the surgeon. That is, the interactive surgical simulation may be a surgical simulation whereby there are certain ‘branch points’ in the simulation which depend upon actions taken by the surgeon within the simulation (e.g. selecting a first or second option). The actions taken by the networked surgeons when attempting the interactive surgical simulation therefore have consequences on the outcome of the surgery (or surgical event) within the interactive simulation. In turn, further decisions may be required to be taken by the networked surgeons based upon the consequence of their original decision.

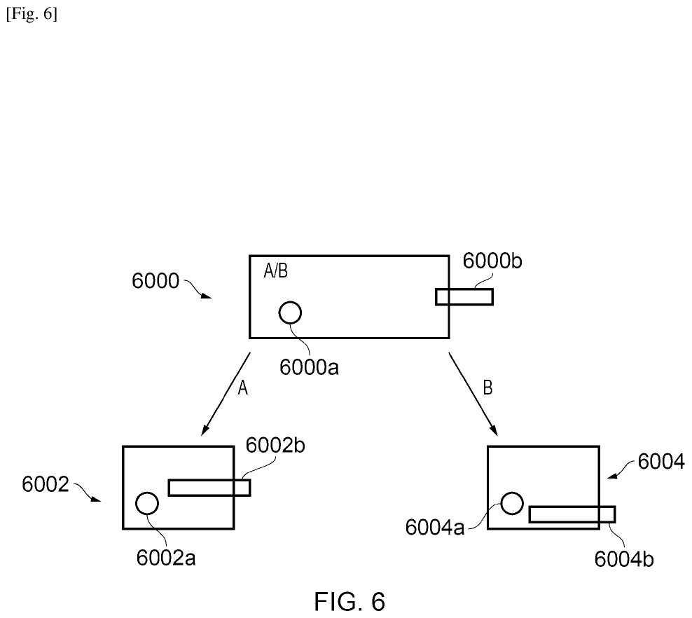

FIG. 6 illustrates an example of an interactive surgical simulation in accordance with embodiments of the disclosure. This surgical simulation illustrated in FIG. 6 of the present disclosure illustrates how an interactive surgical simulation may be experienced by a user (being one of the networked surgeons 3004a, 3004b, or 3004c, for example).

In particular, in this example, a first static 2D image 6000 is provided to the user at a first stage during the interactive surgical simulation. This may be displayed on a display screen of a computing device, for example. The first static 2D image 6000 corresponds to a first juncture of the interactive surgical simulation (such as the first Event Label 4000 in FIG. 4 of the present disclosure). In examples, the first juncture may correspond to the portion of interest which has been identified in the upcoming surgery.

In this example illustrated with reference to FIG. 6, a bleed 6000a has occurred during the interactive surgical simulation. Within the interactive surgical simulation, the ‘operating surgeon’ is equipped with a cauterizing tool 6000b.

At this stage of the interactive surgical simulation, the user is presented with a choice of two options for cauterizing the bleed. The user can either select option ‘A’ (corresponding to performing the cauterization at a first location relative to the bleed) or the user can select option ‘B’ (corresponding to performing the cauterization at a second location relative to the bleed). The user may select an option within the interactive surgical simulation using an input device such as user input device 1006 described with reference to FIG. 1 of the present disclosure, for example.

Once the user has made a selection, the interactive surgical simulation will proceed based on that selection. That is, if the user selected option A, the ‘operating surgeon’ within the interactive surgical simulation will perform the cauterization at the first location relative to the bleed and the second static 2D image 6002 will be displayed to the user. Within this second static image, the cauterizing tool is displayed at a location 6002b relative to the bleed (6002a). However, if the user selected option ‘B’ the interactive surgical simulation will proceed to the third static 2D image 6004. Within this third static 2D image 6004, the cauterizing tool is displayed at the second location 6004b relative to the bleed 6004a.

In some examples, the effectiveness of the virtual ‘operating surgeons’ actions to cauterize the bleed may vary depending on whether option ‘A’ or option ‘B’ was selected by the user. Accordingly, further options may be provided to the user depending on which option they selected when image 6000 was displayed.

Once the user has navigated through a complete branch of the interactive surgical simulation, the interactive surgical simulation may end. The choices and decisions made by the user as they navigated through the interactive simulation may be recorded in performance data which can later be retrieved by the system 2000. This is explained in more detail below.

Since the interactive simulation includes the portion of interest of the upcoming surgery, it can be ensured that the performance data of the networked surgeons when attempting the interactive surgical simulation will be advantageous in the development of a validated portion of interest and/or a validated surgical simulation of the upcoming surgery. In other words, the interactive surgical simulation experienced by the networked surgeons is tailored to the upcoming surgery.

It will be appreciated that the interactive surgical simulations of the present disclosure are not particularly limited to the example provided in FIG. 6 of the present disclosure. Rather, the interactive surgical simulations may include a sequence of 2D static images, a stream of video data, or a number of virtual reality environments which include, at least, the portion of interest of the upcoming surgery. In particular, the simulation may include a realistic representation of a surgical scenario which corresponds to the portion of interest of the upcoming surgery, a simulated camera viewpoint of the portion of interest of the upcoming surgery, data relating to the operation (such as patient monitoring data) or the like.

In order to provide a relevant interactive surgical simulation to the network of surgeons (being an interactive surgical simulation including, at least, the portion of interest identified in the upcoming surgery) the circuitry of system 2000 may be configured to retrieve an interactive surgical simulation from a database and modify the interactive surgical simulation based on the surgical information of the upcoming surgery.

That is, in some examples, a database of pre-produced interactive surgical simulations may be accessible by system 2000. Then, once the portion of interest for the upcoming surgery has been identified, the system 2000 may retrieve a corresponding interactive surgical simulation from the database (being an interactive surgical simulation which contains the portion of interest). This interactive surgical simulation may then be provided to the network of surgeons.

Furthermore, the system 2000 may be configured to adapt the pre-produced interactive surgical simulation depending on the surgical information which has been received. For example, the system 2000 may adapt the appearance of the surgical simulation, the outcome of certain decisions and/or the relative probabilities of certain events occurring depending on the surgical information of the upcoming surgery.

In other examples, the providing unit 2004 may be configured to generate an interactive simulation simulating the portion of interest of the upcoming surgery in a format which may be viewed, and interacted with, by networked surgeons.

That is, the system 2000 (and, in particular, the providing unit 2004) may be configured to generate a simulation including the portion of interest of the surgical event and further add one or more interactive elements to the simulation in order to produce an interactive surgical simulation. This newly generated interactive surgical simulation may be provided to the network of surgeons. In this manner, the providing unit 2004 may augment surgical simulations with one or more interactive elements which can be selected by a user.

In certain examples, the surgical simulations may be generated by a trained surgical simulation model. That is, a simulation training system may be provided to train the surgical simulation model by creating a training dataset of past surgical data.

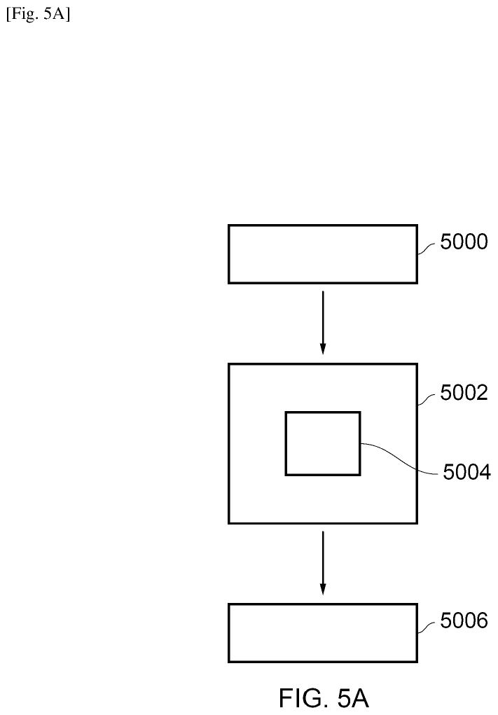

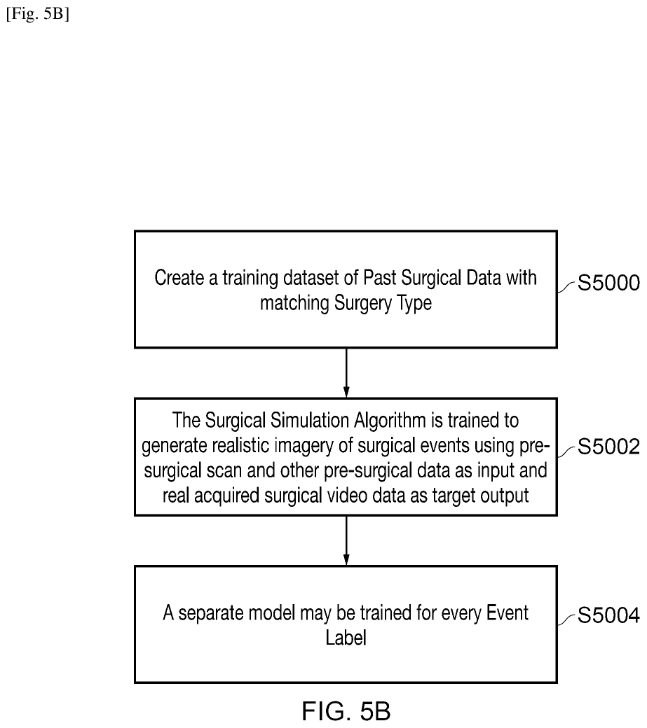

FIG. 5A of the present disclosure illustrates a system which can be used to train a surgical simulation model and generate a surgical simulation of the upcoming surgery (including the portion of interest of the upcoming surgery).

In this example system of FIG. 5A, a database of past surgical procedures 5000 is provided. This may be included as part of the database 3000 illustrated with reference to FIG. 3 of the present disclosure. When training the model, past surgical data is extracted or retrieved from this database 5000 by system 2000. This data is received by the simulation training system 5002 (which may, itself, be part of system 2000).

The dataset of past surgical data is then split into a training set and a test set by the simulation training system 5002. An 80% to 20% split may be used for the training set and test set respectively. However, the split into a training set and a test set is not particularly limited in this regard, and any percentage split between the training set and test set may be used as required depending on the situation.

A surgical simulation unit 5004 may then use information, such as pre-surgical scan data and corresponding Event Labels in the training set portion of the past surgical data to generate still images or short video representations of each key event within the past surgical data.

Generated image data is then scored by its closeness (in terms of pixel value similarity) to the ground truth image data from the past surgery (being actual visual data of the past surgical procedures). The closer the prediction of the appearance of the surgical event is to the actual visual data of the past surgical procedure, the higher the score.

According to the values of these scores across the whole past surgical database, certain weights and parameters of the surgical simulation unit are adjusted. The process is then iteratively repeated by the surgical simulation unit 5004.

In examples, this weighting adjustment may follow standard statistical improvement methods such as the Monte Carlo method. However, the present disclosure is not particularly limited in this regard.

The training process may be ended or completed after a fixed number of iterations (e.g. 1000), or alternatively or in addition, based on a threshold requirement for the model (e.g. when the success scores do not improve over several iterations). The surgical simulation unit 5004 may then be tested on the test set to determine success scores when the trained model is applied to ‘unseen’ surgical data of the test set.

Once trained in this manner, the surgical simulation unit 5004 may be configured to produce a surgical simulation 5006 (such as still images or a short video representation) for key events within an upcoming surgery based on the information such as the pre-surgical scan data and corresponding Event Labels.

Optionally, an identical training phase may be performed by the simulation training system 5002 based on patient sensor data or other data which will be displayed as part of the Interactive Surgical Simulation. In this case, patient sensor data is the target data of the simulation unit, and pre-surgical data is the source.

In certain situations, the simulation training system 5002 may be implemented as a machine learning system. In particular, deep learning models may be used (as an example of a machine learning system). These deep learning models are constructed using neural networks. These neural networks include an input layer and an output layer. A number of hidden layers are located between the input layer and the output layer. Each layer includes a number of individual nodes. The nodes of the input layer are connected to the nodes of the first hidden layer. The nodes of the first hidden layer (and each subsequent hidden layer) are connected to the nodes of the following hidden layer. The nodes of the final hidden layer are connected to the nodes of the output layer.

In other words, each of the nodes within a layer connect back to all the nodes in the previous layer of the neural network.

Of course, it will be appreciated that both the number of hidden layers used in the model and the number of individual nodes within each layer may be varied in accordance with the size of the training data and the individual requirements in simulating the interactive surgical simulations.

Now, it will be appreciated that each of the nodes takes a number of inputs, and produces an output. The inputs provided to the node (through connections with the previous layers of the neural network) have weighting factors applied to them.

In a neural network, the input layer receives a number of inputs (which can include surgical information such as pre-surgical scan data). These inputs are then processed in the hidden layers, using weights that are adjusted during the training. The output layer then produces a prediction from the neural network (such as a simulation of the upcoming surgery).

Specifically, during training, the training data may be split into inputs and targets. The input data is all the data except from the target (being the upcoming surgery which the simulation unit 5004 is trying to predict). The input data is then analysed by the neural network during training in order to adjust the weights between the respective nodes of the neural network. In examples, the adjustment of the weights during training may be achieved through linear regression models. However, in other examples, non-linear methods may be implemented in order to adjust the weighting between nodes to train the neural network.

Effectively, during training, the weighting factors applied to the nodes of the neural network are adjusted in order to determine the value of the weighting factors which, for the input data provided, produces the best match to the target data. That is, during training, both the inputs and target outputs are provided. The network then processes the inputs and compares the resulting output against the target data (such as an image or scene of the actual historic surgical event). Differences between the output and the target data are then propagated back through the neural network, causing the neural network to adjust the weights of the respective nodes of the neural network.

Of course, the number of training cycles (or epochs) which are used in order to train the model may vary in accordance with the situation. In some examples, the model may be continuously trained on the training data until the model produces an output within a predetermined threshold of the target data.

Once trained, new input data can then be provided to the input layer of the neural network, which will cause the model to generate (on the basis of the weights applied to each of the nodes of the neural network during training) a predicted output for the given input data.

Of course, it will be appreciated that the present embodiment is not particularly limited to the deep learning models (such as the neural network) and any such machine learning algorithm can be used in accordance with embodiments of the disclosure depending on the situation.

In this manner, surgical simulations of the upcoming surgery may be generated by the surgical simulation unit 5004 of system 2000.

Once the simulation has been generated, the system 2000 (and, in particular, the providing unit 2004) may overlay certain interactive features on top of the surgical simulation to create the interactive surgical simulation. These features may include one or more buttons or interactive features through which the networked surgeon may make choices about the progression of the scenario, user interface (UI) features to modify the viewpoint (e.g. pinch to zoom, or two finger swipes to pan the camera), UI features to allow the networked surgeon to give feedback on the simulation (e.g. a thumbs up/down icon which can be pressed during or after the simulation), or the like.

The interactive elements which are included within the surgical simulation may vary in their number and complexity depending on the type of surgery which is being simulated and the desired complexity of the interactive surgical simulation.

In particular, the interactive elements may correspond to the Event Labels which have been identified in the surgical data, with each Event Label leading to one or more further branches in the surgical simulation.

As described with reference to FIG. 3 of the present disclosure, the interactive surgical simulation which has been generated may be provided over a network 3004 to a group of surgeons connected to the network 3004a, 3004b and 3004c.

Alternatively, a database may store the generated interactive surgical simulations for later provision to the network of surgeons.

In examples, the network of surgeons may include a group of surgeons which have subscribed to an interactive surgical simulation service. These surgeons may be located at a number of different medical facilities. In this case, the interactive surgical simulation may be provided (e.g. transmitted) via network 3004 to a registered device of each of those surgeons.

However, in other cases, the interactive surgical simulation may be uploaded to a central server or the like which is accessible by each of the surgeons in the network of surgeons. Then, each of the surgeons may access the central server in order to retrieve the interactive surgical simulation. Each surgeon may then obtain access to the central server through the provision of a web link or address, for example.

Turning now to FIG. 5B of the present disclosure, an example method of a training phase for training the surgical simulation unit 5004 of system 2000 is shown. The surgical simulation unit 5004 may, in some examples, be implemented as a surgical simulation algorithm which is trained to generate realistic imagery of surgical events. The example method of FIG. 5B illustrates a method of training the surgical simulation algorithm in this situation.

The example method of FIG. 5B starts at step S5000.

In step S5000, the method comprises creating a training dataset of past surgical data with matching surgery type. The training dataset of past surgical data may be stored in a database of past surgical procedures such as database 5000 illustrated with reference pre-surgical to FIG. 5A of the present disclosure. The past surgical data may include scan data or other data which was available before the surgery (e.g. patient data) and/or video data which was available before the surgery. Moreover, past surgical data may therefore consist of video data sections associated with surgery type, operating surgeon identity, and/or semantic labels which are assigned to points in time or temporal sections within the video data. In some examples, the past surgical data used as training data may be limited to data which matches the surgery type of the upcoming surgery. That is, if the upcoming surgery is a particular type of surgery (such as a certain type of colonoscopy procedure) then the past surgical data used as training data may be limited to past surgical data obtained from this particular type of surgery. This ensures that the data used as training data is most relevant for the upcoming surgery and thus improves the accuracy of interactive simulation which is produced.

In examples, the training data may include two distinct portions: the input data, being data obtained before the past surgery (i.e. pre-surgical scan data) and target data, being data obtained during or after the past surgery (i.e. video acquired during the surgery). The input data of the training data can then be used to train the model to predict the target data of the training data.

In the example method of FIG. 5B, the training dataset of past surgical data is split into a training set and a test set. This may be an 80% to 20% split between the training data and the test set, for example. However, the present disclosure is not particularly limited to this ratio and any percentage split between the training set and test set may be used. The training set of the past surgical data is then used in order to train the model, while the test set is used in order to test and verify the trained model against the past surgical data. Verification of the trained model on the past surgical data in this manner may improve the accuracy of the interactive simulation which is produced.

The method then proceeds to step S5002. In step S5002, the surgical simulation algorithm is trained to generate realistic imagery of surgical events using pre-surgical scan and other pre-surgical data as input and real acquired surgical data as target output. Specifically, the surgical simulation algorithm uses the input data of the training set of the past surgical data (such as past-surgical scan data and event labels) to generate still images or short video representations of the surgical event. The surgical simulation algorithm may be implemented as a deep learning model or a machine learning model as described with reference to FIG. 5A of the present disclosure. The generated image data may then be scored by its closeness in terms of pixel value similarity to the ground truth image data from the past surgery (i.e. the corresponding target data of the training data of the past surgical data). According to the success scores of the generated image data across the whole past surgical database (i.e. across the entirety of the training data), the parameters of the surgical simulation algorithm are adjusted. This adjustment may be performed by a Monte-Carlo method or any other standard statistical improvement method. Once the parameters of the surgical simulation algorithm have been adjusted, the process of generating still images or short video representations of the surgical event may be repeated for the training data using the adjusted surgical simulation algorithm. The success scores of the generated image data may again be determined, and the parameters of the surgical simulation algorithm adjusted accordingly.

The training process may be ended after a set number of iterations (e.g. 1000 iterations or the like). However, the present disclosure is not limited in this respect. Alternatively, the training process may be repeated until a target level of success scores are achieved and/or until the success scores do not show any further improvement over several iterations.

Once the training process has been performed, the trained surgical simulation algorithm may then be tested on the test set to determine performance scores for the model. The performance scores indicate how well the trained model is able to predict the target data for the test data of the past surgical data. If the performance scores achieve a satisfactory level, then the trained model is ready to be used on the upcoming surgery. However, if the trained model does not achieve satisfactory scores on the test data, then further training of the model is required before it can be used for the upcoming surgery. This may include training the model on an increased set of training data and/or increasing the number of training iterations, for example.

Then, as illustrated in FIG. 5B of the present disclosure, the method may proceed to step S5004. Here, the training phase may be repeated for all, or a subset of, Event Labels in the past surgical database. That is, even for a given upcoming surgery type (e.g. a colonoscopy procedure) there may be several different Event Labels within the upcoming surgery (corresponding to different stages of the procedure, as explained in detail with reference to FIG. 4 of the present disclosure). Therefore, in some examples, it may be advantageous to train the model (i.e. the surgical simulation algorithm) independently for each Event Label of the surgical type, such that the model is tailored to each type of Event Label which may occur in the upcoming surgery. This may further improve the accuracy of the surgical simulation which is produced using the trained model.

Accordingly, in step S5004, a separate model may be trained for every event label.

Optionally, an identical training phase as is described with reference to FIG. 5B of the present disclosure may be performed for an algorithm to simulate patient sensor data or other data which will be displayed as part of the Interactive Surgical Simulation. In this case, patient sensor data is the target data of the simulation algorithm, and Pre-surgical data is the source. The process flow for training the model is the same as described with reference to FIG. 5B of the present disclosure in this case.

It will be appreciated that the manner of providing the interactive surgical simulation to the network of surgeons is not particularly limited to these examples and any such method may be used in accordance with embodiments of the present disclosure as required.

<Receiving Unit>

As described with reference to FIG. 2 of the present disclosure, the receiving unit 2006 is configured to receive performance data from the network of surgeons, the performance data being indicative of actions taken by one or more surgeons in response to the interactive surgical simulation.

The receiving unit 2006 may be any type of receiving device which is capable of receiving the performance data from the network of surgeons, such as a network connection 1008 described with reference to FIG. 1 of the present disclosure, for example.

In the present disclosure, performance data may include any information which is produced as the surgeons interact with the interactive simulation. In particular, the performance data may be indicative of decisions made in terms of events within the simulation (e.g. to cauterize or suture a wound), a technique used within the simulation (e.g. the location of a cauterizing action relative to a target bleed location), the type of tool or tools used within the interactive simulation (e.g. whether the surgeon chooses to use a first or second type of tool) and data regarding the performance of the surgeon within the simulation (e.g. the speed of the surgeons reactions and decisions at various stages within the interactive simulation).

The performance data may also include information regarding the interaction of the surgeon with the simulated patient monitoring data. That is, an action or actions taken by the networked surgeon in response to the output of certain types of patient monitoring data (e.g. the blood pressure of the patient) may also be recorded and included in the performance data. The performance data may further indicate the type of patient monitoring data which the networked surgeon finds most useful during the simulation. That is, the performance data may indicate types the surgeon selected to view at each stage of the simulation.

Performance data may be available from the user input device used by the networked surgeon as they attempt the interactive surgical simulation (such as unit input device 1006). Touch interaction data may indicate the patient monitoring data which the networked surgeon selects to view within the simulation, for example.

Alternatively, other techniques, such as gaze monitoring or the like, may also identify the data which surgeon finds the most useful during the simulation.

As explained with reference to FIGS. 5A, 5B and 6 of the present disclosure, the simulation which is provided to the network of surgeons may include a video of the surgical environment or a virtual environment. In this case, the performance data may also include the input and actions taken by the surgeon to reposition the camera (viewpoint) within the surgical environment during the surgical procedure. That is, a surgeon may position the camera (viewpoint) within the surgical environment during the surgical procedure in order to improve the view of a region of interest within the surgical scene (such as a bleed). The performance data may therefore indicate the preferred camera position of the surgeon for each stage of the surgical procedure. This information may be useful for instructing the operating surgeon of the upcoming surgery the best location from which to view the surgical scene. Moreover, this information may be useful to instruct a robotic control device the best location to position a robotic device (such as a camera) during the upcoming surgical procedure.

Moreover, the performance data collected as the networked surgeons attempt the interactive simulation may also include information which more generally provides feedback regarding the surgical simulation. That is, at the end of the interactive surgical simulation, the surgeon may be requested to provide feedback on elements such as how realistic the surgical simulation appears and/or an overall rating of the surgical simulation (including an estimated difficulty factor or the like). Other feedback information may also be provided by the surgeon depending on the type of the surgical simulation. This feedback information may be provided as part of the performance data once the surgeon has completed the surgical simulation. Alternatively, this feedback may be provided by the surgeon as they complete each stage of the surgical simulation.

However, it will be appreciated that the specific type of performance data is not limited to the above examples and can include any information obtainable from the interactive surgical simulation as that simulation is experienced by each of the individual networked surgeons.

In this regard, it will be appreciated that the performance data is not limited to information which is received from a single surgeon's attempt at the interactive surgical simulation. Rather, the interactive surgical simulation is provided to a network of surgeons (e.g. surgeons 3004a, 3004b and 3004c as described with reference to FIG. 3 of the present disclosure), such that the performance data is indicative of each surgeon's individual attempt at the interactive surgical simulation as they interact with the interactive surgical simulation. This crowdsourced performance data, from the network of surgeons, thus enables the system 2000 to analyze in detail the different approaches taken by the surgeons to the interactive surgical simulation. From this performance data, the ‘best practice’ response to the critical event in the portion of interest of the simulation can be determined.

In order for the performance data to be recorded or obtained, each surgeon of the network of surgeons will attempt the interactive surgical simulation on a respective electronic device such as a smartphone, personal computing device, tablet computing device, laptop computer device or the like. The individual performance data of each of the networked surgeons can then be recorded. In some examples, it may be advantageous for networked surgeons to attempt the interactive surgical simulation on a device which includes a touch screen interface or the like. Use of a touch screen interface to attempt the interactive surgical simulation may further improve the sense of realism and immersion for the networked surgeon. This will further enhance the applicability of the validated surgical simulation to the upcoming surgery.

The actions, inputs and decisions taken by each of the networked surgeons will then be recorded individually during the interactive surgical simulation to produce the performance data of that surgeon for the interactive surgical simulation. It will be appreciated that the interactive simulation need not be completed or attempted simultaneously by each of the networked surgeons.

In some examples, the performance data may be compiled and stored locally on the surgeon's personal device. The receiving unit 2006 may then receive the performance data for each surgeon directly from the surgeon's personal device. Alternatively, the performance data for each surgeon may be compiled and stored on a central database. The receiving unit 2006 may then receive the performance data for all surgeons directly from the central database. This may be advantageous in a situation where the networked surgeons do not attempt the surgical simulation at the same time, as the receiving unit 2006 of system 2000 need only make a single access request to the central database to retrieve the performance data for all the networked surgeons.

In some examples, there may be a time limit for completion of the interactive surgical simulation. Only performance data compiled before the expiry of this time limit will be received by the receiving unit 2006. This may be advantageous when there is limited time available prior to the upcoming surgery.

In some examples, each surgeon may be able to attempt the interactive surgical simulation a plurality of times. This enables each surgeon to fine-tune their response to the situations encountered in the interactive surgical simulation. Accordingly, each surgeon may develop their optimal approach to the situation encountered in the interactive surgical simulation. This enables the surgeon to develop their skill and improves the performance data received by the system 2000. Moreover, in examples, the surgeon may choose to restrict the provision of performance data to that a subset of the attempts at the interactive surgical simulation.

However, in other examples, it may be advantageous to limit the number of times the surgeon can actually attempt each interactive surgical simulation. In examples, each surgeon may be allowed only a single attempt at the interactive surgical simulation. This may help ensure that the performance data received by system 2000 is not biased by the performance data of a surgeon who makes repeated attempts at the interactive surgical simulation.

<Validating Unit>

Once the performance data has been received by the receiving unit 2006, the validating unit 2008 of system 2000 is configured to validate at least one of the portion of interest and/or the interactive surgical simulation based on the received performance data.

As explained with reference to the receiving unit 2006 of system 2000, the performance data is data indicative of each individual networked surgeon's attempt (or attempts) at the interactive surgical simulation. However, in some examples, it may be advantageous for the performance data of the individual surgeons to be combined to create a single processed performance data (the combined performance data).

In this regard, the validation unit 2008 may be configured in order to extract and combine certain information from the performance data which has been received. This information may consist of the most frequently selected surgical decision for each Event Label or juncture within the interactive surgical simulation. That is, if 90% of the surgeons who attempted the interactive surgical simulation made a first decision at a first Event Label, while only 10% of surgeons made an alternative decision at that same Event Label, it may be determined that the decision made by the 90% of surgeons is the best decision to be made at that first Event Label. The identification of the best decision may be made based upon an analysis of the mode or median of certain parameters of the collective performance data received from the networked surgeons, for example. However, the mathematical operation used in order to identify the most likely surgical decision in the case of each ‘Event Label’ or juncture within the interactive surgical simulation is not particularly limited in this regard.

The combined performance data may also include indications of the most used surgical data types or patient monitoring data types for each individual stage or Event Label of the surgical simulation. In some examples, this may be the top two data types for example. Of course, this number may change depending on the number of data types which are possible or recommended for display during the interactive surgical simulation. In this manner, the combined performance data may therefore indicate that, when there is a bleed, most surgeons wish to see data regarding the patient's blood pressure, for example.

The combined performance data may also include identities for the Event Label associated with the interactive surgical simulations which has the most ‘realistic’ or ‘unrealistic’ votes from networked surgeons (based on the feedback information included in the individual performance data). Alternatively, the combined performance data may indicate the median viewpoint settings which were selected by the networked surgeons for each Event Label in the interactive surgical simulation. Furthermore, the combined performance data may indicate the Event Labels which are associated with the greatest uncertainty of the networked surgeons; uncertainty of the networked surgeons may be measured by the variance of the input surgical decisions made in the simulation performance data and/or the median amount of time taken by the surgeons when deciding how to respond once the event associated with the Event Label has occurred.

The combined performance data is not particularly limited to these examples, and may include any data obtained from a collective analysis of the individual performance data received from each surgeon.

The combined performance data (or, optionally, the individual performance data received by the receiving unit 2006 itself) may then be used by the validating unit 2008 in order to validate at least one of the portion of interest and/or the interactive surgical simulation.

In some examples, validation of the interactive surgical simulation may include a determination that the surgical simulation, or individual parts thereof, meet a certain threshold level or realism or approval from the networked surgeons (based on the feedback information provided by the surgeons). Validation may also indicate, or require, a threshold level of convergence of actions or decisions made by the networked surgeons at a certain juncture within the surgical simulation. Portions of the surgical simulation which meet this threshold level may then be validated by the system 2000. In some examples, one or more flags may then be added to appropriate portions of the interactive surgical simulation to indicate which portions of the surgical simulation have been determined as highly useful by the network of surgeons.

Furthermore, in some examples, once validated, visual overlays may then be added to selected portions of the surgical simulation to indicate the best practice or most likely course of action to be taken at a given juncture within the surgical simulation. The operating surgeon viewing the validated simulation (such as surgeon 3002 in the example of FIG. 3 of the present disclosure) may then readily comprehend the best course of action to be taken at a certain stage of the upcoming surgical procedure (i.e. upcoming surgery 3000 in the example of FIG. 3) based on the performance data of the networked surgeons.

In this manner, the system may be configured to validate the surgical simulation when a quality factor indicated by the performance data is above a threshold value.

However, if the surgical simulation, or a portion therefore, is not validated (because it does not meet a required threshold level of convergence, for example) then one or more remedial actions may be taken by system 2000 with regards to that specific portion of the surgical simulation. The type of remedial action taken will vary depending on the reason for lack of validation of the surgical simulation. For example, if a portion of the surgical simulation does not achieve validation for reason of lack of convergence in actions taken by the networked surgeons, remedial action may include providing that portion of the surgical simulation to a wider range of networked surgeons (to increase the number of surgeons who have attempted the surgical simulation). This may then lead to convergence of actions. Alternatively, a number of surgeons who provided eclectic solutions to the interactive surgical simulation may be requested to repeat the surgical simulation with options limited to a restricted number of the most favored actions. Again, this may lead to convergence of response.

Alternatively, if the reasons for lack of validation of the surgical simulation is a lack of realism or approval indicated by the surgeons in feedback information, the system 2000 may perform further processing on the interactive simulation (to improve realism) or may, alternatively, replace or update the interactive elements within the interactive surgical simulation to provide alternative options to the surgeons.

The process of providing the interactive surgical simulation to the network, receiving the performance data from the network and validating the surgical simulation may continue for a number of cycles until the validation unit 2008 validates the interactive surgical simulation.

In this manner, the system 2000 may rectify issues with the surgical simulation of the upcoming surgical procedure before that surgical simulation is provided to the operating surgeon. In other words, only a validated surgical simulation (meeting the approval of the network of surgeons) is provided to the operating surgeon for the upcoming surgery.

Validation of the portion of interest of the surgical simulation may also include an assessment of the performance data of the networked surgeons. For example, if the performance data (or combined performance data) indicates a majority of the networked surgeons find a particular section of the interactive simulation challenging, the system may then recognize that particular section of the surgical simulation as a portion of interest (even if it was not originally identified as such). The interactive surgical simulation may then be adapted according to the new or updated portion of interest, such that further information regarding the best course of action for the new or updated portion of interest may be obtained.

Specifically, once the networked surgeons have created some initial performance data, the initial performance data may be used to alter the probabilities used for the determination of the portion of interest (such as the ‘criticality value’). This may be include determining the proportion of networked surgeons that followed the same decision choices as the operating surgeons in the past surgical data of similar surgical procedures, for example. Accordingly, the system 2000 is able to update and adapt in accordance with latest medical and surgical developments and techniques.

Consider again the example of FIG. 4 of the present disclosure. In this example, 90% of networked surgeons may follow the decisions resulting in Event Label 4000, 4004 and 4008, whereas in past surgery data this may be only 60%. The two different values may be averaged, weighted by the number of participants in each category (that is, the number of past surgeries and number of networked surgeons). The significance value may also be adjusted based on the performance data, where the variance of networked surgeon decisions is normalized to lie between 0 and 1, for example. This value would then be averaged with the significance value to adjust the final criticality value for the Event Label. Portions of interest may then be identified based on the final criticality score for each Event Label. As such, the portion of interest may also be validated and/or updated based upon the performance data received from the network.

Therefore, more generally, in certain examples, the system 2000 may be configured to calculate a weighting for the performance data; apply the weighting to the performance data to obtain a weighted performance data; and validate the portion of interest and/or the interactive surgical simulation using the weighted performance data.

In some examples, once validated, the surgical simulation may be provided to the operating surgeon (being the surgeon who will be performing the upcoming surgery (such as surgeon 3002 in the example of FIG. 3 of the present disclosure)) such that the operating surgeon may review the validated surgical simulation which has been produced by system 2000. This enables the operating surgeon to readily understand the ‘best course of action’ to take if and when certain events occur within the upcoming surgery, based on the performance data received from the networked surgeons. Furthermore, it enables the operating surgeon to understand the most likely events which may occur during the upcoming surgery.

Alternatively, statistical information derived from the performance data (or the combined performance data) may be provided directly to the operating surgeon, once the simulation has been validated, instead of provision of the validated simulation directly to the operating surgeon itself. This may be advantageous in situations where the operating surgeon cannot readily view the validated surgical simulation. For example, the operating surgeon may wish to receive statistical information regarding the actions of the networked surgeons during the surgery.

Furthermore, in some examples, the operating surgeon may attempt the interactive surgical simulation before they are provided with the validated surgical simulation. This enables the operating surgeon to compare how their actions and decisions relate to the actions and decisions of the network of surgeons. In fact, the operating surgeon may attempt or review the validated interactive surgical simulation a number of times prior to the upcoming surgery in order to familiarize themselves with the best course of action to take in the upcoming surgery.

Alternatively or in addition, the validated surgical simulation may be provided to a robotic control system or robotic surgeon. This enables the robotic control system or robotic surgeon to select the best or most beneficial actions to perform during the upcoming surgery. In particular, the best or most beneficial actions may include the subset of actions which were rated as the most effective and efficient by the networked surgeons. Alternatively, or in addition, this may include actions of the robotic surgeon where networked surgeons did not intervene in autonomous action performed by the robotic control surgeon during the surgical simulation. These actions are likely the actions which the operating surgeon for the upcoming surgery will allow the robotic surgeon to perform without intervention. Performance of these actions will improve the efficiency and effectiveness of the actions of the robotic surgeon during the upcoming surgery.

In some examples, certain beneficial actions may be a particular action performed at a particular speed. That is, the respective junctures of the interactive simulation may include options where the robotic control system performs an action (such as cauterizing a bleed) at a different set of speeds (e.g. a fast, medium and slow movement option). The networked surgeons may intervene more frequently within the surgical simulation when the robotic control device moves or controls a robotic arm in order to perform that action at a high speed than compared to the same action performed at a lower speed; this may, particularly, be the case with surgeons who have had less experience working with robotic surgical devices. As such, it may be more efficient for the robotic control device to make autonomous actions, or semi-autonomous actions, at a reduced speed (as this results in fewer interventions from the operating surgeon).

In this manner, robotic motor control functions for the upcoming surgery may be selected based on the validated performance data obtained from the surgical simulation.

In addition, certain control functions of an autonomous scope holder arm, an autonomous tool holder arm and/or an endoscopic support arm may be selected due to scope positions which are rated as advantageous in the validated performance data. This may also include adjustment of the operating parameters of an automated surgical robotic function, such as a camera control function. For example, the camera position settings during the upcoming operation may be determined by the median value (or other mathematical function) of the viewpoint settings selected by the networked surgeons during the interactive surgical simulation.

As such, in examples, system 2000 may be configured to adjust operating parameters of a surgical robot based on the validated portion of interest and/or the validated surgical simulation.

Furthermore, in examples, certain elements of the operating surgeon's display (such as a display screen or augmented reality projection in an operating theatre) may be selected or adapted based on the validated performance data. This may include displaying a warning that the robot will handover control to the operating surgeon with sufficient time for them to act on it, without distracting the surgeon from their other tasks.

In this manner, the validated surgical simulation may improve the efficiency of operation of the robotic control system and facilitate interactions between the operating surgeon and one or more robotic surgeons during surgery.

<Advantageous Technical Effects>

Hence, more generally, a system for validating a surgical simulation is provided by the present disclosure. Validation of the surgical simulation in by the system 2000 enables identification of the most likely and impactful events which may occur during a surgical procedure, thus optimizing a utility of input from a network of surgeons. The best practice response to these events may also be determined ahead of the upcoming surgery based on the crowdsourced performance data received from the network of surgeons.

In particular, provision of interactive surgical simulation to the network of surgeons enables collection of detailed surgeon performance data which can be used to inform the operating surgeon of potential dangers in the upcoming surgery, as well as a range of popular strategies for addressing these dangers. Embodiments therefore support the surgical decision process. In fact, these strategies may include strategies which would not have been considered by the operating surgeon based on their individual analysis of the surgical procedure alone.

Furthermore, the validated surgical simulation may be utilized by the operating surgeon and/or a robotic control system in order to facilitate and improve interactions between the operating surgeon and one or more robotic surgeons or robotic control devices during surgery.

The advantageous technical effects provided by the claimed invention are not particularly limited in this regard.

<Additional Modifications>

In system 2000, described with reference to FIG. 2 of the present disclosure, the performance data of all surgeons is used in order to validate the interactive surgical simulation. This may be based either on the individual performance data of each surgeon or the combined performance data received from the network of surgeons.

However, there may be certain example situations where it would be advantageous that the performance data of one surgeon, or one group of surgeons, was used to validate the interactive surgical simulation (or had a greater influence over the validation of the surgical simulation).

As such, optionally, the system 2000 may be further configured to select the surgeon or surgeons which would provide the most appropriate or relevant feedback for the interactive surgical simulation. The system 2000 may then be configured to validate the surgical simulation based on the performance data received from this selected surgeon or group of surgeons.

In some examples, a value score, indicating how suitable each surgeon or group of surgeons in the network of surgeons is for providing performance data with respect to a certain interactive surgical simulation, may be obtained. The value score may be determined according to the experience (i.e. number of surgeries performed) of each networked surgeon with surgeries of a matching surgery type. The performance data may then be weighted according to this value score.

Alternatively, the system may select a group of surgeons of the networked surgeons who have particular experience performing a certain type of surgery and provide the interactive surgical simulation directly to this selected group of surgeons.

The interactive surgical simulation provided by system 2000 may also be particularly advantageous for training surgeons within the networked group of surgeons, as it provides an interactive simulation of a surgical procedure. Accordingly, in some examples of the present disclosure, the system 2000 may determine a value score for the estimated contribution of the interactive surgical simulation to each individual networked surgeon's personal training targets or simulation exposure targets. These targets may be acquired from an existing training application, for example. The interactive surgical simulation may then be provided to those surgeons who would receive the greatest training benefit from experiencing the interactive surgical simulation. Efficiency of training the individual surgeons of the network of surgeons can therefore be improved.

Furthermore, in some examples, providing unit 2004 of system 2000 may provide the interactive surgical simulation to a robotic control platform, which would enable networked surgeons to interact with the interactive surgical simulation via controls which will correspond to the controls which will be used by the operating surgeon during the upcoming surgery. This is advantageous because the simulation will be tailored more closely to the operating environment of the upcoming surgery.

In further examples, the provision unit 2004 may also provide multiple surgical simulations for each critical juncture, where, for each simulation, a different human robot interaction function may be used. In this example, the networked surgeon may therefore add feedback to the simulation performance data which rates the robot performance during the simulation (e.g. how well it responds and is able to perform the individual requested actions and movement patterns in response to the networked surgeons instructions).

This rating may be then be used by the operating surgeon (such as surgeon 3002) to select certain human robot interaction settings during the upcoming surgery.

<Method>

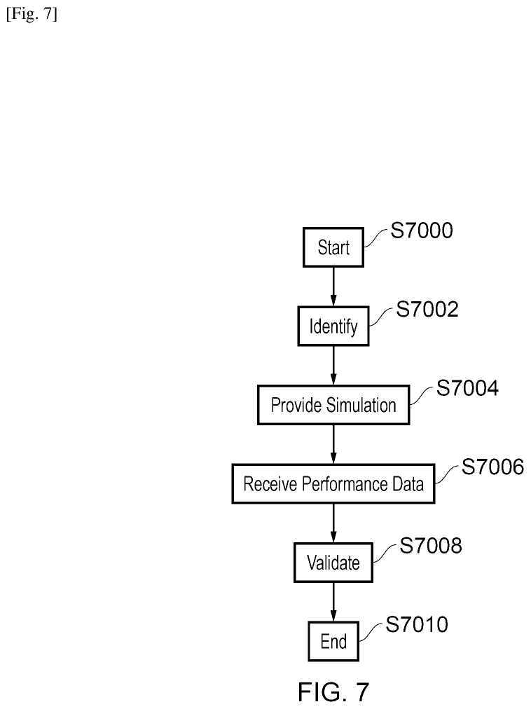

Hence, more generally, a method of validating a surgical simulation is provided in accordance with embodiments of the disclosure. An example method of validating a surgical simulation in accordance with embodiments of the disclosure is illustrated with reference to FIG. 7 of the present disclosure. The method may be performed by a system such as system 2000 illustrated with reference to FIG. 2 of the present disclosure, for example.

The method starts with step S7000, and proceeds to step S7002.

In step S7002, the method includes identifying a portion of interest of a surgical event based on surgical information.

Once the portion of interest has been identified, the method proceeds to step S7004.

In step S7004, the method includes providing an interactive surgical simulation to a network of surgeons, the interactive surgical simulation including the portion of interest of the surgical event.

When the interactive surgical simulation has been provided, the method proceeds to step S7006.

In step S7006, the method includes receiving performance data from the network of surgeons, the performance data being indicative of actions taken by one or more surgeons in response to the interactive surgical simulation.

The method then proceeds to step S7008.

In step S7008, the method includes validating at least one of the portion (or portions) of interest and/or the interactive surgical simulation based on the received performance data.

The method then proceeds to, and ends with, step S7010.

### Example Implementation

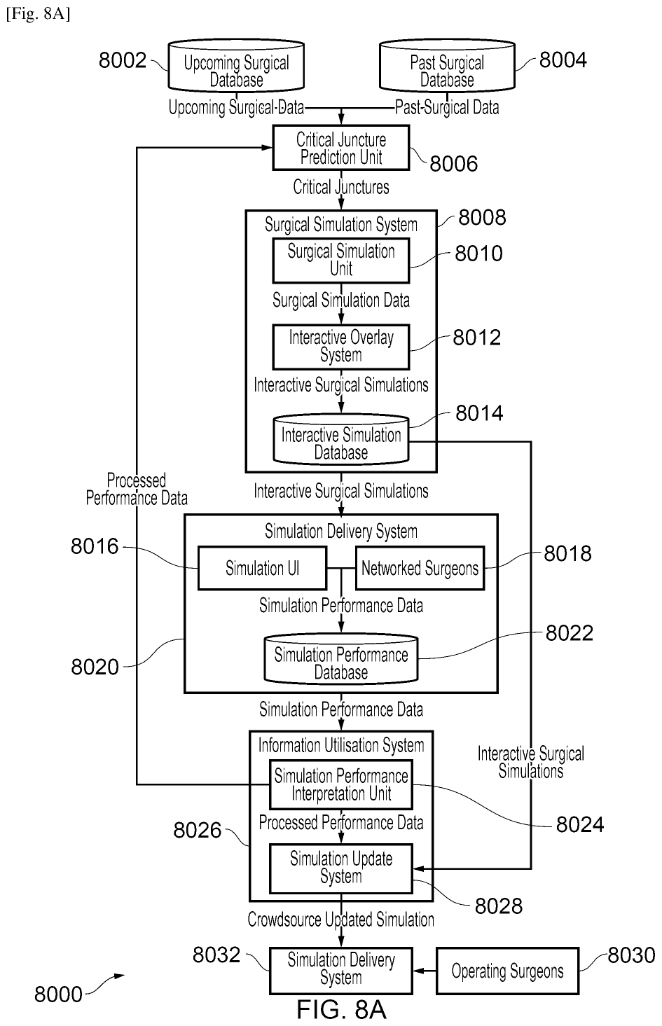

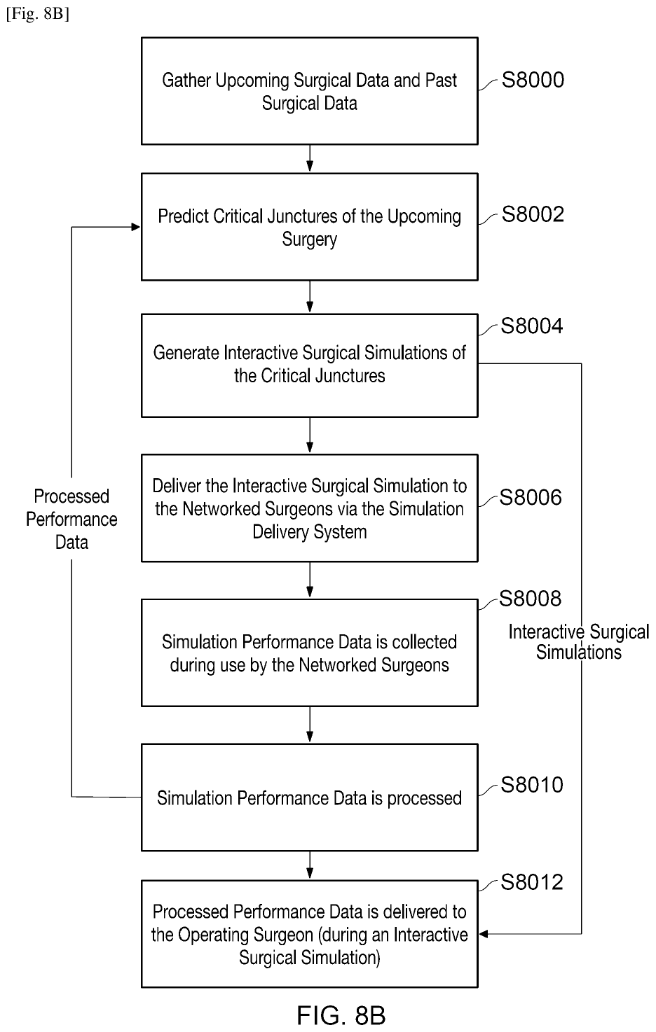

Embodiments of the disclosure may also be arranged in the following example implementations described with reference to FIGS. 8A and 8B of the present disclosure.

FIG. 8A illustrates an example system 8000 in accordance with embodiments of the disclosure.

A database 8002 (The “Upcoming Surgical Database”) of data relating to an upcoming surgery (the “Upcoming Surgical Data”), which has been populated with various relevant data types is provided.

The Upcoming Surgical data includes (but is not limited) to details of the Upcoming Surgery (the “Surgery Type”), patient electronic medical record, patient scan data, data relating to the surgical skills of the operating surgeon (the “operating surgeon data”), or the like. In particular, the details of the upcoming surgery may include the operation identity (e.g. cataracts surgery) and/or details of the operations system to be used (e.g. the surgical robot model). Moreover, the details relating to the surgical skills of the operating surgeon may include surgeon skill level, data relating to decisions made in previous operations (this may be in the form of probabilities of different actions given a stimulus occurrence (e.g. in the event of a bleed in tissue area x, the surgeon is 80% likely to cauterise the bleed themselves, and 20% likely to ask their second surgeon to perform the cauterisation), and/or the surgeon's success with different surgical actions.

The probabilities of different actions given a stimulus occurrence may be pre-calculated from assessments of past surgeries performed by the operating surgeon. Furthermore, the surgeon's success with different surgical actions may be in the form of a percentage error rate with actions of a certain type based on automatic or manual flagging of serious errors in past surgical performance data.

System 8000 also includes a database 8004 (the “Past Surgical Database”) to store data relating to past surgeries, such as video data, surgical notes, or other (The “Past Surgical Data”). In this example the Past Surgical Data primarily consists of video data, the video data may be annotated by existing video processing algorithms to label the events which occur in the video data. In examples, the Past Surgical Data may consist of video data sections associated with surgery type, operating surgeon identity, and semantic labels which are assigned to points in time or temporal sections within the video data (the “Event Labels”). In examples, the Event Labels may include adverse events, such as a bleed, surgeon error, or other event which negatively impacts the surgical outcome; operating surgeon actions, such as to make and incision, or to apply suction; the anatomical location of the event, such as upper colon, or the like.

The Past Surgical Data may be structured and grouped within the database according to the Event Labels and their sequential relationship within the past surgeries (as described in detail with reference to FIG. 4 of the present disclosure). Furthermore, the pre-surgical scan data (and other data relating to each past surgery) may also be included in the Past Surgical Data.

The system 8000 may also include a unit 8006 (the Critical Juncture Prediction unit) which uses Upcoming Surgical Data and Past Surgical Data to predict the situations which may occur during the upcoming surgery which may have a high risk to the surgical outcome or where the outcome of the situation is uncertain (The “Critical Junctures”). The Critical Junctures may be defined semantically in the same format as Event Labels, such as: 1st incision location and/or bleed occurrence during excision of a tumour, for example.

In embodiments, the Critical Juncture Prediction unit may consist of a rules-based algorithm. Additionally, the processed performance later (described in more detail below) may be included in the determination of Critical Junctures once this data is available.

System 8000 further includes a Surgical Simulation system 8008 to simulate the Critical Junctures in a format which may be viewed and interacted with by networked surgeons 8018, such as an interactive video where the surgeon may select actions from a set of programmed options (the “Interactive Surgical Simulation”). The surgical simulation system 8008 may consist of: a Surgical Simulation unit 8010, an Interactive Overlay system 8012 and an Interactive Simulation database 8014.

The Surgical Simulation unit 8010 is configured to generate simulations of the Critical Juncture (which may include a sequence of 2D static images or video data). The simulation may include a realistic representation of a surgical scenario which corresponds to the Event Label, a simulated camera viewpoint and/or realistic data relating to the operation, such as patient monitoring data.

The Interactive Overlay system 8012 is configured to add features to the surgical simulation data in order to create the interactive surgical simulation (the “Interactive Features”). These Interactive Features may include buttons or interactable features through which the networked surgeon may make choices about the progression of the scenario or simulation; user interface (UI) features to modify the viewpoint (e.g. pinch to zoom or two finger swipes to pan the camera); UI features to allow the networked surgeon to give feedback on the simulation, such as a thumbs up/down icon which can be pressed after the simulation to evaluate certain aspects of the simulation. These aspects of the simulation may include scenario or simulation realism and/or outcome.

The Interactive Simulation Database 8014 is configured to store the generated Interactive Surgical Simulations.

System 8000 further includes a Simulation Training system (not show in FIG. 8A) to train the surgical simulation unit 8010 using the past surgical data.

The system 8000 further includes a Simulation Delivery System 8020 to allow the networked surgeons 8018 to view and interact with the Interactive Surgical Simulation and to collect data relating to the networked surgeons performance in the interactive surgical simulation (the “Simulation Performance Data”). The Simulation Delivery System 8020 may include a software defined user interface (the “Simulation UI”) 8016 which enables user input and display via hardware platforms such as smartphones or desktop PCs; a communication network to enable networked surgeons 8018 to receive the Interactive Surgical Simulation.

The performance data which is collected may include decisions made by the networked surgeons 8018 in terms of the semantic choice made by the user (e.g. “Cauterise” or “Suture”); technique used (including location relative to a target structure (such as location of cauterising action relative to a bleed location)); tools used during the simulation; and/or additional data regarding human decision performance such as speed of decisions and/or physiological data in the period prior to a decision being made (e.g. a physiologically relevant period (such as up to 10 seconds)). The performance data which is collected may also include data of the interaction of the networked surgeons 8018 with the simulated patient monitoring data. This may include identities of the data types which the user selected to view (made available from touch interaction data, for example) or may use gaze data. The performance data may also include interactions of the networked surgeons with the positioning of the viewpoint within the simulation. The performance data may also include networked surgeon 8018 feedback regarding the simulation including evaluation of scenario realism and/or rating of scenario utility.

In examples, the Simulation Delivery system 8020 may further consist of a hardware user interface such as a smartphone or the like, with a screen and touch interface; a mechanism for recording user inputs during the Interactive Surgical Simulation; and/or a database (the Simulation Performance Database”) 8022 to store the simulation performance data.

Furthermore, the system 8000 may also include an Information Utilisation System 8026 which is configured to generate preparatory actions which will improve the outcome of the Upcoming Surgery based on the Simulation Performance Data. In examples, this may be a direct communication of performance statistics relating to different Critical Junctures, applied to Interactive Surgical Simulations (The “Crowdsource Updated Simulations”). Hence, the Information Utilisation System 8026 may consist of a Simulation Performance Interpretation unit 8024 to calculate useful information from the Simulation Performance Data (i.e. “Processed Performance Data”). This data may consist of the most likely surgical decision in the case of each Event Label simulation (i.e. the median); identities for the most used surgical data types for each Event Label Simulation (e.g. the top two data types; this number may change depending on certain factors including the number of data types which are possible or recommended to display by either the UI which the operating surgeon 8030 will use during the upcoming surgery and/or the UI of the Simulation Delivery system). The Processed Performance Data may also include Identities for the Event Label associated Interactive Surgical Simulations which have the most ‘unrealistic’ votes from Networked Surgeons; the median viewpoint settings which were selected by the Networked surgeons for each Event Label simulation; and/or the Event Labels which are associated with the greatest uncertainty of the Networked Surgeons. This may be measured by the variance of the input surgical decisions made in the Simulation Performance Data.

Finally, the system 8000 may also include a Simulation Update System 8028 which is configured to update Interactive Surgical Simulations, which may, in examples, include adding Processed Performance Data overlays to interactive Surgical Simulations and/or selecting Interactive Surgical Simulations which are either highly rated by networked surgeons 8018 and/or rated as realistic with a realism level above a pre-defined threshold (e.g. 95% of the networked surgeons rated the simulation as realistic).

The crowdsourced updated simulation may then be passed to a second simulation delivery system 8032 which can be accessed by the operating surgeon 8030 before and/or during the upcoming surgery.

FIG. 8B illustrates an example method which may be performed by an example system such as that illustrated with reference to FIG. 8A of the present disclosure. The example method illustrated in FIG. 8B is an example method of the present disclosure.

The example method starts with step S8000.

In step S8000, the Upcoming Surgical Database described with reference to FIG. 8A of the present disclosure is populated with Upcoming Surgical Data. In examples, this may be performed through a surgical planning software platform, where details of an upcoming surgery are manually input by users.

Then, in step S8002, the Critical Juncture Prediction unit predicts the Critical Junctures of the Upcoming Surgery. The Critical Juncture Prediction unit may, in examples, extract Past Surgical Data from the Past Surgical Database which matches with Upcoming Surgical Data on a set of pre-defined key parameters. These matching parameters may consist of Upcoming Surgery Type but may also include other data such as Operating Surgeon Data. Matching the Upcoming Surgical Data with the Past Surgical Database based on Operating Surgeon Data may be particularly advantageous in surgical scenarios where the parameters of the Operating Surgeon are important to the possible faults that may occur during a surgery. For example, surgeon skill level may have a high impact on the outcome of the most difficult surgeries and should therefore be used in order to predict the most likely faults in this regard.

In examples, the selected Past Surgical Data may be statistically analysed in order to determine the likelihood of the Different Event Labels for the Upcoming Surgery. This probability may be defined as the proportion of matched past surgeries which contain each Event Label. Probabilities for events may be defined on a 0-1 scale. The probability may then be averaged with a pre-defined ‘significance value’ for each Event Label, which may also be a value of between 0 and 1. For example, an Event Label of a bleed occurrence in the upper colon may have a likelihood of 0.1 and a significance of 0.5. The combined ‘criticality value’ would therefore be 0.3. Other mathematical functions may also be used such as multiplication. The number of most critical (likely and significant) may then selected based on a pre-defined useful number, for example, this may be the top five most critical.

Additionally, once Networked Surgeons have created some Simulation Performance Data, processed Performance Data may be used to alter the probabilities which have been calculated. This may be included by determining the proportion of Networked Surgeons that followed the same decision choices as the Operating Surgeons in the Past Surgical Data. For example, 90% of Networked Surgeons may have followed the decisions resulting in a set of event labels comprising Event Label 1, 3 and 5, whereas, in past surgery data, this may be only 60%. Furthermore, the two values may be averaged, weighted by the number of participants in each category (number of past surgeries and number of Networked Surgeons).

The significance value which has been calculated may also be adjusted by the processed Performance Data, where the variance of Networked Surgeon decisions are normalised to lie between 0 and 1. This value would then be averaged with the significance value to adjust the final criticality score for the Event Label.

Furthermore, in some embodiments, the Upcoming Surgery Type may have multiple options, such as options of different robotic surgical platforms which may be used. In these examples, different sets of Critical Junctures may be created for the different surgical platform options.

In step S8004, the Surgical Simulation System creates an Interactive Surgical Simulation of the Critical Juncture where the Upcoming Surgical Data, and Critical Juncture Event Labels are input into the Surgical Simulation unit to generate Surgical Simulation Data. The Interactive Overlay System then adds Interactive Features to the Surgical Simulation Data. In some examples, this may include the step of adding a user interface (UI) overlay to the end of a Surgical Simulation Data segment (corresponding to an Event Label) which prompts the user to make a choice. The displayed choice may be selected from the Event Labels present in Past Surgical Data, which follow on in time from the Event Label in past surgeries of matching Surgery Type.

Then, in step S8006, the Simulation Delivery System displays the Interactive Surgical Simulation to the Networked Surgeons.

In step S8008, the method comprise collecting Simulation Performance Data as the networked surgeons interact with the Simulation UI. This data is stored in the Simulation Performance Database. The Simulation Performance is described in more detail with reference to FIG. 8A of the present disclosure.

In step S8010, the simulation Performance data is processed. In examples, the Information Utilisation System may create Crowdsource Updated Simulations of the Upcoming Surgery, where the Simulation Performance Interpretation unit perform statistical analysis (i.e. determining medians and other statistical functions) of the Simulation Performance Data. Furthermore, flags are added to appropriate Interactive Surgical Simulations within the Interactive Simulation Database, which have been determined as highly useful. Moreover, UI overlays are then added to Interactive Surgical Simulations selected by the Simulation Update System.

Finally, in step S8012, the Simulation Delivery System may then be used to deliver the Crowdsource Updated Simulation to the Operating Surgeon.

While certain example implementations of the present disclosure have been described with reference to FIGS. 8A and 8B of the present disclosure, it will be appreciated that the present disclosure is not particularly limited in this regard. Rather, the present disclosure can be applied more general as described with reference to the device, method and computer program product for validating a surgical simulation described with reference to FIGS. 1 to 7 of the present disclosure.

### Example Surgical Systems

The above described embodiments of the disclosure are applicable to a number of example surgical systems.

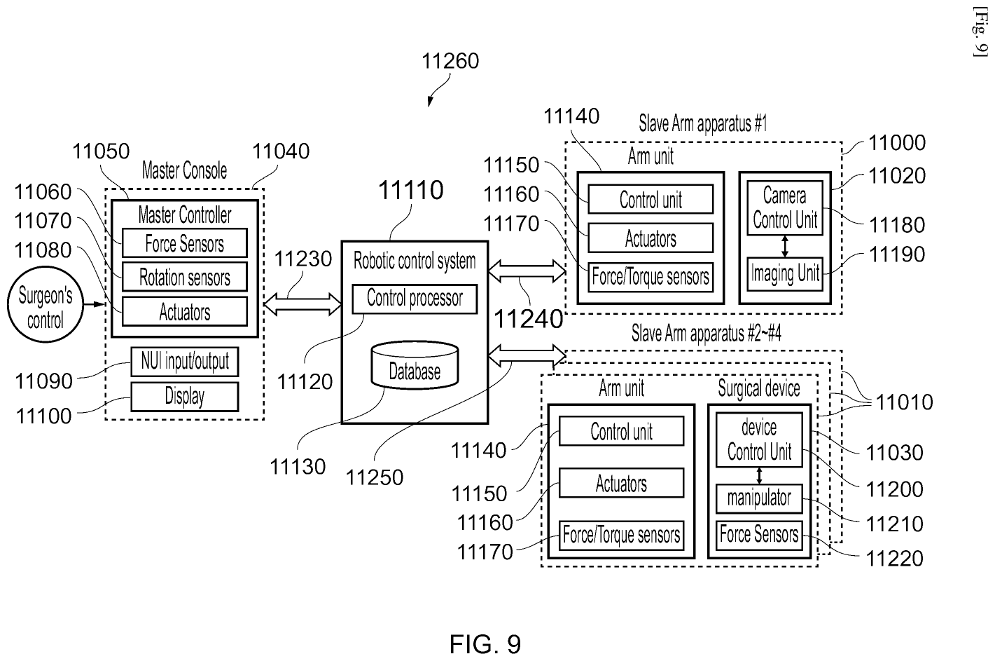

FIG. 9 schematically shows an example of a computer assisted surgery system 11260 to which the present technique is applicable. The computer assisted surgery system is a master slave system incorporating an autonomous arm 11000 and one or more surgeon-controlled arms 11010. The autonomous arm holds an imaging device 11020 (e.g. a medical scope such as an endoscope, microscope or exoscope). The one or more surgeon-controlled arms 11010 each hold a surgical device 11030 (e.g. a cutting tool or the like). The imaging device of the autonomous arm outputs an image of the surgical scene to an electronic display 11100 viewable by the surgeon. The autonomous arm autonomously adjusts the view of the imaging device whilst the surgeon performs the surgery using the one or more surgeon-controlled arms to provide the surgeon with an appropriate view of the surgical scene in real time.

The surgeon controls the one or more surgeon-controlled arms 11010 using a master console 11040. The master console includes a master controller 11050. The master controller 11050 includes one or more force sensors 11060 (e.g. torque sensors), one or more rotation sensors 11070 (e.g. encoders) and one or more actuators 11080. The master console includes an arm (not shown) including one or more joints and an operation portion. The operation portion can be grasped by the surgeon and moved to cause movement of the arm about the one or more joints. The one or more force sensors 11060 detect a force provided by the surgeon on the operation portion of the arm about the one or more joints. The one or more rotation sensors detect a rotation angle of the one or more joints of the arm. The actuator 11080 drives the arm about the one or more joints to allow the arm to provide haptic feedback to the surgeon. The master console includes a natural user interface (NUI) input/output for receiving input information from and providing output information to the surgeon. The NUI input/output includes the arm (which the surgeon moves to provide input information and which provides haptic feedback to the surgeon as output information). The NUI input may also include a voice input, a line of sight input and/or a gesture input. The master console includes the electronic display 11100 for outputting images captured by the imaging device 11020.

The master console 11040 communicates with each of the autonomous arm 11000 and one or more surgeon-controlled arms 11010 via a robotic control system 11110. The robotic control system is connected to the master console 11040, autonomous arm 11000 and one or more surgeon-controlled arms 11010 by wired or wireless connections 11230, 11240 and 11250. The connections 11230, 11240 and 11250 allow the exchange of wired or wireless signals between the master console, autonomous arm and one or more surgeon-controlled arms.

The robotic control system includes a control processor 11120 and a database 11130. The control processor 11120 processes signals received from the one or more force sensors 11060 and one or more rotation sensors 11070 and outputs control signals in response to which one or more actuators 11160 drive the one or more surgeon controlled arms 11010. In this way, movement of the operation portion of the master console 11040 causes corresponding movement of the one or more surgeon controlled arms.

The control processor 11120 also outputs control signals in response to which one or more actuators 11160 drive the autonomous arm 11000. The control signals output to the autonomous arm are determined by the control processor 11120 in response to signals received from one or more of the master console 11040, one or more surgeon-controlled arms 11010, autonomous arm 11000 and any other signal sources (not shown). The received signals are signals which indicate an appropriate position of the autonomous arm for images with an appropriate view to be captured by the imaging device 11020. The database 11130 stores values of the received signals and corresponding positions of the autonomous arm.

For example, for a given combination of values of signals received from the one or more force sensors 11060 and rotation sensors 11070 of the master controller (which, in turn, indicate the corresponding movement of the one or more surgeon-controlled arms 11010), a corresponding position of the autonomous arm 11000 is set so that images captured by the imaging device 11020 are not occluded by the one or more surgeon-controlled arms 11010.

As another example, if signals output by one or more force sensors 11170 (e.g. torque sensors) of the autonomous arm indicate the autonomous arm is experiencing resistance (e.g. due to an obstacle in the autonomous arm's path), a corresponding position of the autonomous arm is set so that images are captured by the imaging device 11020 from an alternative view (e.g. one which allows the autonomous arm to move along an alternative path not involving the obstacle).

It will be appreciated there may be other types of received signals which indicate an appropriate position of the autonomous arm.

The control processor 11120 looks up the values of the received signals in the database 11130 and retrieves information indicating the corresponding position of the autonomous arm 11000. This information is then processed to generate further signals in response to which the actuators 11160 of the autonomous arm cause the autonomous arm to move to the indicated position.

Each of the autonomous arm 11000 and one or more surgeon-controlled arms 11010 includes an arm unit 11140. The arm unit includes an arm (not shown), a control unit 11150, one or more actuators 11160 and one or more force sensors 11170 (e.g. torque sensors). The arm includes one or more links and joints to allow movement of the arm. The control unit 11150 sends signals to and receives signals from the robotic control system 11110.

In response to signals received from the robotic control system, the control unit 11150 controls the one or more actuators 11160 to drive the arm about the one or more joints to move it to an appropriate position. For the one or more surgeon-controlled arms 11010, the received signals are generated by the robotic control system based on signals received from the master console 11040 (e.g. by the surgeon controlling the arm of the master console). For the autonomous arm 11000, the received signals are generated by the robotic control system looking up suitable autonomous arm position information in the database 11130.

In response to signals output by the one or more force sensors 11170 about the one or more joints, the control unit 11150 outputs signals to the robotic control system. For example, this allows the robotic control system to send signals indicative of resistance experienced by the one or more surgeon-controlled arms 11010 to the master console 11040 to provide corresponding haptic feedback to the surgeon (e.g. so that a resistance experienced by the one or more surgeon-controlled arms results in the actuators 11080 of the master console causing a corresponding resistance in the arm of the master console). As another example, this allows the robotic control system to look up suitable autonomous arm position information in the database 11130 (e.g. to find an alternative position of the autonomous arm if the one or more force sensors 11170 indicate an obstacle is in the path of the autonomous arm).

The imaging device 11020 of the autonomous arm 11000 includes a camera control unit 11180 and an imaging unit 11190. The camera control unit controls the imaging unit to capture images and controls various parameters of the captured image such as zoom level, exposure value, white balance and the like. The imaging unit captures images of the surgical scene. The imaging unit includes all components necessary for capturing images including one or more lenses and an image sensor (not shown). The view of the surgical scene from which images are captured depends on the position of the autonomous arm.

The surgical device 11030 of the one or more surgeon-controlled arms includes a device control unit 11200, manipulator 11210 (e.g. including one or more motors and/or actuators) and one or more force sensors 11220 (e.g. torque sensors).

The device control unit 11200 controls the manipulator to perform a physical action (e.g. a cutting action when the surgical device 11030 is a cutting tool) in response to signals received from the robotic control system 11110. The signals are generated by the robotic control system in response to signals received from the master console 11040 which are generated by the surgeon inputting information to the NUI input/output 11090 to control the surgical device. For example, the NUI input/output includes one or more buttons or levers included as part of the operation portion of the arm of the master console which are operable by the surgeon to cause the surgical device to perform a predetermined action (e.g. turning an electric blade on or off when the surgical device is a cutting tool).

The device control unit 11200 also receives signals from the one or more force sensors 11220. In response to the received signals, the device control unit provides corresponding signals to the robotic control system 11110 which, in turn, provides corresponding signals to the master console 11040. The master console provides haptic feedback to the surgeon via the NUI input/output 11090. The surgeon therefore receives haptic feedback from the surgical device 11030 as well as from the one or more surgeon-controlled arms 11010. For example, when the surgical device is a cutting tool, the haptic feedback involves the button or lever which operates the cutting tool to give greater resistance to operation when the signals from the one or more force sensors 11220 indicate a greater force on the cutting tool (as occurs when cutting through a harder material, e.g. bone) and to give lesser resistance to operation when the signals from the one or more force sensors 11220 indicate a lesser force on the cutting tool (as occurs when cutting through a softer material, e.g. muscle). The NUI input/output 11090 includes one or more suitable motors, actuators or the like to provide the haptic feedback in response to signals received from the robot control system 11110.

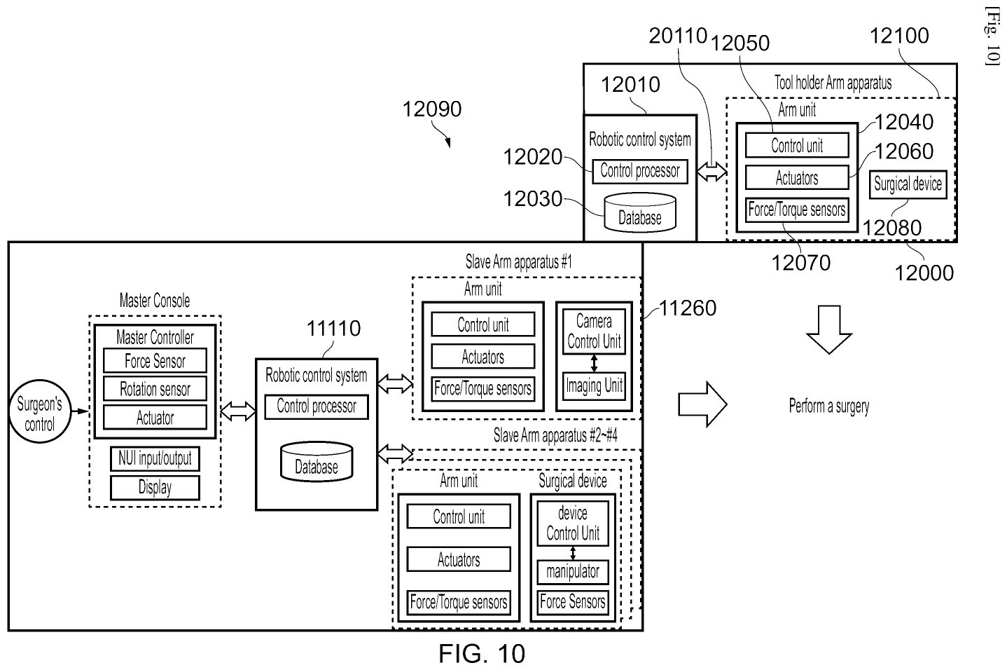

FIG. 10 schematically shows another example of a computer assisted surgery system 12090 to which the present technique is applicable. The computer assisted surgery system 12090 is a surgery system in which the surgeon performs tasks via the master slave system 11260 and a computerised surgical apparatus 12000 performs tasks autonomously.

The master slave system 11260 is the same as FIG. 9 and is therefore not described. The system may, however, be a different system to that of FIG. 9 in alternative embodiments or may be omitted altogether (in which case the system 12090 works autonomously whilst the surgeon performs conventional surgery).

The computerised surgical apparatus 12000 includes a robotic control system 12010 and a tool holder arm apparatus 12100. The tool holder arm apparatus 12100 includes an arm unit 12040 and a surgical device 12080. The arm unit includes an arm (not shown), a control unit 12050, one or more actuators 12060 and one or more force sensors 12070 (e.g. torque sensors). The arm includes one or more joints to allow movement of the arm. The tool holder arm apparatus 12100 sends signals to and receives signals from the robotic control system 12010 via a wired or wireless connection 12110. The robotic control system 12010 includes a control processor 12020 and a database 12030. Although shown as a separate robotic control system, the robotic control system 12010 and the robotic control system 11110 may be one and the same. The surgical device 12080 has the same components as the surgical device 11030. These are not shown in FIG. 10.

In response to control signals received from the robotic control system 12010, the control unit 12050 controls the one or more actuators 12060 to drive the arm about the one or more joints to move it to an appropriate position. The operation of the surgical device 12080 is also controlled by control signals received from the robotic control system 12010. The control signals are generated by the control processor 12020 in response to signals received from one or more of the arm unit 12040, surgical device 12080 and any other signal sources (not shown). The other signal sources may include an imaging device (e.g. imaging device 11020 of the master slave system 11260) which captures images of the surgical scene. The values of the signals received by the control processor 12020 are compared to signal values stored in the database 12030 along with corresponding arm position and/or surgical device operation state information. The control processor 12020 retrieves from the database 12030 arm position and/or surgical device operation state information associated with the values of the received signals. The control processor 12020 then generates the control signals to be transmitted to the control unit 12050 and surgical device 12080 using the retrieved arm position and/or surgical device operation state information.

For example, if signals received from an imaging device which captures images of the surgical scene indicate a predetermined surgical scenario (e.g. via neural network image classification process or the like), the predetermined surgical scenario is looked up in the database 12030 and arm position information and/or surgical device operation state information associated with the predetermined surgical scenario is retrieved from the database. As another example, if signals indicate a value of resistance measured by the one or more force sensors 12070 about the one or more joints of the arm unit 12040, the value of resistance is looked up in the database 12030 and arm position information and/or surgical device operation state information associated with the value of resistance is retrieved from the database (e.g. to allow the position of the arm to be changed to an alternative position if an increased resistance corresponds to an obstacle in the arm's path). In either case, the control processor 12020 then sends signals to the control unit 12050 to control the one or more actuators 12060 to change the position of the arm to that indicated by the retrieved arm position information and/or signals to the surgical device 12080 to control the surgical device 12080 to enter an operation state indicated by the retrieved operation state information (e.g. turning an electric blade to an “on” state or “off” state if the surgical device 12080 is a cutting tool).

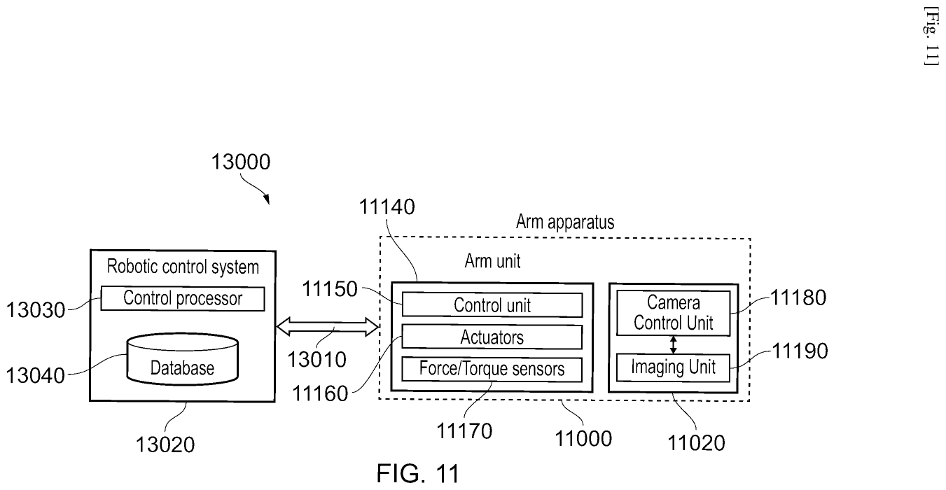

FIG. 11 schematically shows another example of a computer assisted surgery system 13000 to which the present technique is applicable. The computer assisted surgery system 13000 is a computer assisted medical scope system in which an autonomous arm 11000 holds an imaging device 11020 (e.g. a medical scope such as an endoscope, microscope or exoscope). The imaging device of the autonomous arm outputs an image of the surgical scene to an electronic display (not shown) viewable by the surgeon. The autonomous arm autonomously adjusts the view of the imaging device whilst the surgeon performs the surgery to provide the surgeon with an appropriate view of the surgical scene in real time. The autonomous arm 11000 is the same as that of FIG. 9 and is therefore not described. However, in this case, the autonomous arm is provided as part of the standalone computer assisted medical scope system 13000 rather than as part of the master slave system 11260 of FIG. 9. The autonomous arm 11000 can therefore be used in many different surgical setups including, for example, laparoscopic surgery (in which the medical scope is an endoscope) and open surgery.

The computer assisted medical scope system 13000 also includes a robotic control system 13020 for controlling the autonomous arm 11000. The robotic control system 13020 includes a control processor 13030 and a database 13040. Wired or wireless signals are exchanged between the robotic control system 13020 and autonomous arm 11000 via connection 13010.

In response to control signals received from the robotic control system 13020, the control unit 11150 controls the one or more actuators 11160 to drive the autonomous arm 11000 to move it to an appropriate position for images with an appropriate view to be captured by the imaging device 11020. The control signals are generated by the control processor 13030 in response to signals received from one or more of the arm unit 11140, imaging device 11020 and any other signal sources (not shown). The values of the signals received by the control processor 13030 are compared to signal values stored in the database 13040 along with corresponding arm position information. The control processor 13030 retrieves from the database 13040 arm position information associated with the values of the received signals. The control processor 13030 then generates the control signals to be transmitted to the control unit 11150 using the retrieved arm position information.

For example, if signals received from the imaging device 11020 indicate a predetermined surgical scenario (e.g. via neural network image classification process or the like), the predetermined surgical scenario is looked up in the database 13040 and arm position information associated with the predetermined surgical scenario is retrieved from the database. As another example, if signals indicate a value of resistance measured by the one or more force sensors 11170 of the arm unit 11140, the value of resistance is looked up in the database 12030 and arm position information associated with the value of resistance is retrieved from the database (e.g. to allow the position of the arm to be changed to an alternative position if an increased resistance corresponds to an obstacle in the arm's path). In either case, the control processor 13030 then sends signals to the control unit 11150 to control the one or more actuators 1116 to change the position of the arm to that indicated by the retrieved arm position information.

Embodiments of the disclosure may also be arranged in accordance with the following numbered clauses:

(1)

A device for validating a surgical simulation, the device including circuitry configured to:
identify a portion of interest of a surgical event based on surgical information;
provide an interactive surgical simulation to a network of surgeons, the interactive surgical simulation including the portion of interest of the surgical event;
receive performance data from the network of surgeons, the performance data being indicative of actions taken by one or more surgeons in response to the interactive surgical simulation; and
validate at least one of the portion of interest and/or the interactive surgical simulation based on the received performance data.

(2)

The device according to Clause (1), wherein the circuitry is configured to obtain at least a portion of the surgical information from a database, the surgical information including information of at least one of an upcoming surgical event, a current surgical event and/or a previous surgical event.

(3)

The device according to Clause (1) or (2), wherein the circuitry is further configured to control one or more sensors and/or devices to obtain at least a portion of the surgical information.

(4)

The device according to Clause (2) or (3), wherein the surgical information includes information of at least one of a surgical type, patient record, a type of surgical robot used in the surgical event and/or a type of surgical equipment.

(5)

The device according to any preceding Clause, wherein the circuitry is further configured to: retrieve an interactive surgical simulation from a database; modify the interactive surgical simulation based on the surgical information; and provide the interactive surgical simulation to the network of surgeons.

(6)

The device according to any preceding Clause, wherein the circuitry is further configured to generate a simulation including the portion of interest of the surgical event; add one or more interactive elements to the simulation in order to produce an interactive surgical simulation; and provide the interactive surgical simulation to the network of surgeons.

(7)

The device according to Clause (6), wherein the circuitry is configured to generate the simulation including the portion of interest of the surgical event based on a trained model.

(8)

The device according to Clause (6) or (7), wherein the interactive elements include one or more virtual buttons which can be used to control the progression of the surgical simulation, one or more virtual buttons to modify the viewpoint of the surgical simulation, and one or more feedback elements which can be used in order to provide feedback on the surgical simulation.

(9)

The device according to any preceding Clause, wherein the performance data includes information of decisions taken by the surgeons in response to interactive elements of the interactive surgical simulation, interactions of the surgeons with the position of the viewpoint in the simulation, rating of the surgical simulation and/or performance metric information of the surgical simulation.

(10)

The device according to any preceding Clause, wherein the portion of interest is a portion of the surgical event identified as a risk to a surgical outcome, a portion of the surgical event where a surgical outcome is uncertain, and/or a portion of the surgical event requiring human interaction machine interaction.

(11)

The device according to any preceding Clause, wherein the interactive surgical simulation includes a plurality of images, video data and/or virtual environments.

(12)

The device according to any preceding Clause, wherein the identification of the portion of interest and/or the provision of the interactive surgical simulation is based on a trained model.

(13)

The device according to any preceding Clause, wherein the circuitry is configured to calculate a weighting for the performance data; apply the weighting to the performance data to obtain a weighted performance data; and validate the portion of interest and/or the interactive surgical simulation using the weighted performance data.

(14)

The device according to any preceding Clause, wherein the circuitry is configured to validate the surgical simulation when a quality factor indicated by the performance data is above a threshold value.

(15)

The device according to any preceding Clause, wherein the circuitry is configured to provide the validated surgical simulation to a surgeon.

(16)

The device according to any preceding Clause, wherein the circuitry is configured to adjust operating parameters of a surgical robot based on the validated portion of interest and/or the validated surgical simulation.

(17)

The device according to any preceding Clause, wherein the circuitry is further configured to update the at least one of the portion of interest and/or the surgical simulation to generate updated content; and provide the updated content to the network of surgeons, when a quality factor indicated by the performance data is below a threshold value.

(18)

The device according to any preceding Clause, wherein the circuitry is further configured to provide the validated interactive surgical simulation to a surgeon, robotic control device or surgical robot operating in the surgical event.

(19)

The device according to any preceding Clause, wherein validating at least one of the portion of interest and/or the interactive surgical simulation includes updating at least one of the portion of interest and/or the interactive surgical simulation.

(20)

A method of validating a surgical simulation, the method comprising:
identifying a portion of interest of a surgical event based on surgical information;
providing an interactive surgical simulation to a network of surgeons, the interactive surgical simulation including the portion of interest of the surgical event;
receiving performance data from the network of surgeons, the performance data being indicative of actions taken by one or more surgeons in response to the interactive surgical simulation; and
validating at least one of the portion of interest and/or the interactive surgical simulation based on the received performance data.

(21)

A computer program product comprising instructions which, when the program is executed by a computer, cause the computer to perform a method of validating a surgical simulation, the method comprising:
identifying a portion of interest of a surgical event based on surgical information;
providing an interactive surgical simulation to a network of surgeons, the interactive surgical simulation including the portion of interest of the surgical event;
receiving performance data from the network of surgeons, the performance data being indicative of actions taken by one or more surgeons in response to the interactive surgical simulation; and
validating at least one of the portion of interest and/or the interactive surgical simulation based on the received performance data.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. A device for validating a surgical simulation, the device including circuitry configured to: identify a portion of interest of a surgical event based on surgical information;
provide an interactive surgical simulation to a network of surgeons, the interactive surgical simulation including the portion of interest of the surgical event;
receive performance data from the network of surgeons, the performance data being indicative of actions taken by one or more surgeons in response to the interactive surgical simulation; and
validate at least one of the portion of interest and/or the interactive surgical simulation based on the received performance data.

2. The device according to claim 1, wherein the circuitry is configured to obtain at least a portion of the surgical information from a database, the surgical information including information of at least one of an upcoming surgical event, a current surgical event and/or a previous surgical event.

3. The device according to claim 1, wherein the circuitry is further configured to control one or more sensors and/or devices to obtain at least a portion of the surgical information.

4. The device according to claim 2, wherein the surgical information includes information of at least one of a surgical type, patient record, a type of surgical robot used in the surgical event and/or a type of surgical equipment.

5. The device according to claim 1, wherein the circuitry is further configured to: retrieve an interactive surgical simulation from a database; modify the interactive surgical simulation based on the surgical information; and provide the interactive surgical simulation to the network of surgeons.

6. The device according to claim 1, wherein the circuitry is further configured to generate a simulation including the portion of interest of the surgical event; add one or more interactive elements to the simulation in order to produce an interactive surgical simulation; and provide the interactive surgical simulation to the network of surgeons.

7. The device according to claim 6, wherein the circuitry is configured to generate the simulation including the portion of interest of the surgical event based on a trained model.

8. The device according to claim 6, wherein the interactive elements include one or more virtual buttons which can be used to control the progression of the surgical simulation, one or more virtual buttons to modify the viewpoint of the surgical simulation, and one or more feedback elements which can be used in order to provide feedback on the surgical simulation.

9. The device according to claim 1, wherein the performance data includes information of decisions taken by the surgeons in response to interactive elements of the interactive surgical simulation, interactions of the surgeons with the position of the viewpoint in the simulation, rating of the surgical simulation and/or performance metric information of the surgical simulation.

10. The device according to claim 1, wherein the portion of interest is a portion of the surgical event identified as a risk to a surgical outcome, a portion of the surgical event where a surgical outcome is uncertain, and/or a portion of the surgical event requiring human interaction machine interaction.

11. The device according to claim 1, wherein the interactive surgical simulation includes a plurality of images, video data and/or virtual environments.

12. The device according to claim 1, wherein the identification of the portion of interest and/or the provision of the interactive surgical simulation is based on a trained model.

13. The device according to claim 1, wherein the circuitry is configured to calculate a weighting for the performance data; apply the weighting to the performance data to obtain a weighted performance data; and validate the portion of interest and/or the interactive surgical simulation using the weighted performance data.

14. The device according to claim 1, wherein the circuitry is configured to validate the surgical simulation when a quality factor indicated by the performance data is above a threshold value.

15. The device according to claim 1, wherein the circuitry is configured to provide the validated surgical simulation to a surgeon.

16. The device according to claim 1, wherein the circuitry is configured to adjust operating parameters of a surgical robot based on the validated portion of interest and/or the validated surgical simulation.

17. The device according to claim 1, wherein the circuitry is further configured to update the at least one of the portion of interest and/or the surgical simulation to generate updated content; and provide the updated content to the network of surgeons, when a quality factor indicated by the performance data is below a threshold value.

18. The device according to claim 1, wherein the circuitry is further configured to provide the validated interactive surgical simulation to a surgeon, robotic control device or surgical robot operating in the surgical event.

19. The device according to claim 1, wherein validating at least one of the portion of interest and/or the interactive surgical simulation includes updating at least one of the portion of interest and/or the interactive surgical simulation.

20. A method of validating a surgical simulation, the method comprising:
identifying a portion of interest of a surgical event based on surgical information;
providing an interactive surgical simulation to a network of surgeons, the interactive surgical simulation including the portion of interest of the surgical event;
receiving performance data from the network of surgeons, the performance data being indicative of actions taken by one or more surgeons in response to the interactive surgical simulation; and
validating at least one of the portion of interest and/or the interactive surgical simulation based on the received performance data.

21. A non-transitory computer program product comprising instructions which, when by a computer, cause the computer to perform a method of validating a surgical simulation, the method comprising:
identifying a portion of interest of a surgical event based on surgical information;
providing an interactive surgical simulation to a network of surgeons, the interactive surgical simulation including the portion of interest of the surgical event;
receiving performance data from the network of surgeons, the performance data being indicative of actions taken by one or more surgeons in response to the interactive surgical simulation; and
validating at least one of the portion of interest and/or the interactive surgical simulation based on the received performance data.
